(19) United States
(12) Patent Application Publication
(10) Pub. No.: US 2017/0105646 A1
(43) Pub. Date: Apr. 20, 2017

(54) MULTI-PARAMETER SENSOR SYSTEM FOR MEASURING PHYSIOLOGICAL SIGNALS

(71) Applicant: BIOPEAK CORPORATION, Ottawa (CA)

(72) Inventors: Alan Bryenton, Ottawa (CA); Izmail Batkin, Ottawa (CA); Neil Webster, Constance Bay (CA)

(21) Appl. No.: 15/390,315

(22) Filed: Dec. 23, 2016

Related U.S. Application Data
(63) Continuation of application No. PCT/CA2015/050598, filed on Jun. 26, 2015.
(60) Provisional application No. 62/017,523, filed on Jun. 26, 2014.

(51) Int. Cl. A61B 5/0408; A61B 5/00; A61B 5/053; A61B 5/04

(52) U.S. Cl. CPC A61B 5/04085; A61B 5/04012; A61B 5/4266; A61B 5/6801; A61B 5/0531

(57) ABSTRACT

Systems, methods and devices relating to a physiological sensor system to be worn on a subject's skin to measure, process or store one or more physiological parameters, the system comprising: at least one pair of sensing electrodes that measure, when in contact with the subject's skin, physiological signals indicative of the one or more physiological parameters; first and second reference electrodes that concurrently provide a source signal and a reference signal for said sensing electrodes in their measurement of the one or more physiological parameters; and a subject-securing device for securing said sensing electrodes and said first and second reference electrodes against the subject's skin in operation.

### CROSS REFERENCE TO RELATED APPLICATIONS

The presently disclosed subject matter is a continuation of and claims the benefit of PCT/CA2015/050598, filed Jun. 26, 2015, which claims the benefit of U.S. Provisional Patent Application Ser. No. 62/017,523, filed Jun. 26, 2014, the disclosures of which are incorporated herein by reference in their entireties.

### FIELD OF THE DISCLOSURE

The present disclosure relates to physiological sensors, and in particular, to a multi-parameter sensor system for measuring physiological signals.

### BACKGROUND

Traditional surface physiological electrodes work as transducers for transforming skin potentials into electrical voltages. Typically, the detected potential and currents are small (voltage V is less or in the order of 10 microV, and current I is less than or in the order of nA) while unrelated physiological electrical noise (i.e. non-signal or artifact) can reach several hundred mV. There are several sources of electrical artifacts in bioelectrical measurements, which may impact the quality of the results.

For example, the salt ion diffusion through the skin surface creates a so-called double layer and corresponding potentials can reach several tens of mV. The skin deformation changes ion distribution of the skin surface that generates low frequency deformation electrical potentials (or deformation potentials) which is much higher than typical ECG voltages.

The contact between body electrolytes and the surface of a solid conductive electrode also generates half-cell potentials of the order of several hundreds mV. The potential between solid conductor and body electrolyte is another source of strong motion artifact.

The high impedance of the stratum corneum is another source of electrical artifact.

The delivery of weak electrical signals from electrodes to a high impedance detecting system is a strong source of noise due to stray capacitance and the tri-bielectricity of cables, environmental electromagnetic fields and embedded static charges. These factors are very sensitive to body motion.

When multiple signals are measured, one or more measurement subsystems can inject unwanted signals into other measurement subsystem, e.g. bio-impedance measurement into ECG and/or sweat.

Improper placement of separate electrodes for sensing multiple signals in an attempt to minimize electromagnetic (EM) susceptibility between the multiple sensors impacts the sensor signal quality due to insufficient physical space on the body to properly place the electrodes.

Existing techniques used to reduce these artifacts include skin preparation by abrading, using electrolytic gel, conditioning of the skin by passing electrical energy or heating, real time impedance control systems, application of high impedance active electronics attached to the electrodes, mechanical stabilization of electrodes, puncturing the skin with micro needles, and application of an additional sensor for motion detection.

There are several drawbacks associated with the use of traditional electrodes. The abrasive skin preparation and puncturing involves discomfort, prolong procedure time and require operator skill whereas the body will heal micro-punctures, skin abrasions and re-grow hair causing signal degradation and thus cannot be used for long term home or hospital applications. The gel electrode application causes signal degradation as a result gel drying, itching, and the possibility of rash from allergic skin reaction and cause considerable discomfort and damage on removal. The use of algorithms to remove the motion artifact can lead to erasing portions of important physiological information. The use of micro needles, heating or passing electrical energy leads to discomfort, skin damage and time-dependent impedance instability. Electrode placement can be difficult due to common or interfering placement requirements of separate measurement subsystems, related susceptibility and signal degradation etc.

Other problems associated with multi-parameter physiological sensors include an increased power consumption and footprint that is problematic on portable and multi-use sensor systems.

This background information is provided to reveal information believed by the applicant to be of possible relevance. No admission is necessarily intended, nor should be construed, that any of the preceding information constitutes prior art.

### SUMMARY

The following presents a simplified summary of the general inventive concept(s) described herein to provide a basic understanding of some aspects of the invention. This summary is not an extensive overview of the invention. It is not intended to restrict key or critical elements of the invention or to delineate the scope of the invention beyond that which is explicitly or implicitly described by the following description and claims.

A need exists for a multi-parameter sensor system for measuring physiological signals that overcome some of the drawbacks of known techniques, or at least, provide a useful alternative thereto. Some aspects of this disclosure provide examples of such sensor systems.

The disclosed subject matter, therefore, according to an exemplary aspect, provides for a physiological sensor system to be worn on a subject's skin to measure one or more physiological parameters, the system comprising: at least one pair of sensing electrodes that measure, when in contact with the subject's skin, physiological signals indicative of the one or more physiological parameters; first and second reference electrodes that concurrently provide a source signal and a reference signal for said sensing electrodes in their measurement of the one or more physiological parameters; and a subject-securing device for securing said sensing electrodes and said first and second reference electrodes against the subject's skin in operation.

The disclosed subject matter, according to another exemplary aspect, provides for a multi-parameter sensor system for measuring physiological signals in a subject, the bioelectrical signals being indicative of physiological parameters, which in some cases is susceptible to non-signal artifacts, the system, in this exemplary aspect, comprising a plurality of electrodes configured to concurrently provide multiple functionalities, such functionalities may include a measurement of physiological signals, a reduction of non-signal artifacts, a current source, a current sink, reference electrode(s), the system further comprising: at least one pair of sensing electrodes that measure physiological signals indicative of physiological parameters, the physiological parameters may include body bio-impedance (possibly in cases where the reference electrodes are acting as a signal source by driving a signal across the body of the subject), ECG, skin-electrode impedance (possibly in cases where the reference electrodes are not acting as a signal source), and sweat-induced, sweat-dependent or other sweat-related physiological signals (possibly when the sensing electrodes are individually cooperating with the conducting electrodes); first and second reference electrodes that concurrently provide (1) a signal source for the first and second sensing electrodes in their measurement of one or more of the physiological parameters and (2) a reference for the sensing electrodes, possibly for any two or more combinations of other electrodes during measurement of the body bio-impedance, skin-electrode bio-impedance, ECG, sweat sensors, acoustic sensors, light sensors, thermal sensors, motion sensors, radiation sensors and other signals measured or detected by the multi-parameter sensor system; and a subject-securing device for securing the sensing electrodes and the reference electrodes against an outer surface of the subject. As such, physiological signals, and other non-physiological signals (which may or may not be related to such physiological signals may be measured) including accelerometry and radiation. These signals may be used on their own or in connection with physiological signals, in either case to provide information relating to the subject.

In accordance with another exemplary aspect of the disclosed subject matter, there is provided a method of concurrently measuring distinct physiological parameters from a subject using a same pair of sensing electrodes and an associated pair of grounding reference electrodes, each in contact with the subject's skin to define a measurement site therebetween, the method comprising: collecting physiological signals via said pair of sensing electrodes; splitting said collected bioelectrical signals in accordance with distinct frequency domains respectively associated with each of the distinct physiological parameters; and switching the reference electrodes between an active mode in which a current is provided across the measurement site and a passive mode in which said current is not provided, wherein said active mode and said passive mode cause the sensing electrodes to assess respective ones of said distinct physiological parameters.

In accordance with another exemplary aspect of the disclosed subject matter, there is provided a method of concurrently measuring a plurality of physiological signals in a subject, the physiological signals being indicative of physiological parameters, but which may in some cases be susceptible to non-signal artifacts, using a plurality of electrodes, each electrode configured to provide at least two concurrent functionalities and being affixed on a subject facing side of a subject securing device, the concurrent functionalities comprising two or more of the following functionalities: measuring at least one physiological signals, reducing non-signal artifacts, providing a current source, providing a current sink, providing a reference electrode, the method comprising: collecting physiological signals across a circuit that includes the subject when a first sensing electrode and a second sensing electrode from the plurality of electrodes are in contact therewith; splitting the physiological signals into one or more processing circuits each configured to assess at least one physiological parameter based on the physiological signals by filtering said physiological signals into separate frequency domains, each of the frequency domains being associated with at least one of the physiological parameters; and switching a third and fourth electrodes from the plurality of electrodes between a first mode for providing current across the measurement site of the first and second sensing electrodes and a second mode wherein current is not provided across the measurement site, wherein the first and second modes cause the first and second sensing electrodes to assess different physiological parameters.

In accordance with another exemplary aspect of the disclosed subject matter, there is provided a physiological sensor system to be worn on a subject's skin to assess one or more physiological parameters, the system comprising: first and second electrodes that measure, when in contact with the subject's skin, physiological signals indicative of the one or more physiological parameters, the output of at least one of the first and second electrodes being split into first and second processing circuits, the first processing circuit optimized for ECG signal processing, the second processing circuit optimized for bio-impedance signal processing; a third electrode and a fourth electrode that may concurrently (i) independently provide a voltage reference, and (ii) together provide a signal for said first and second electrodes; and a subject-securing device for securing said first, second, third and fourth electrodes against the subject's skin in operation.

In accordance with another exemplary aspect of the disclosed subject matter, there is provided a multi-parameter sensor system for measuring physiological signals in a subject, the physiological signals being indicative of physiological parameters, which may in some cases be susceptible to non-signal artifacts, the system comprising a plurality of electrodes configured to concurrently provide multiple functionalities, which may in embodiments comprise of measuring at least one bioelectrical signals, reducing non-signal artifacts, providing a current source, providing a current sink, and providing a reference, the system comprising: at least a first pair of electrodes that concurrently measure at least two physiological signals indicative of at least two of the following physiological parameters: body bio-impedance, skin-electrode bio-impedance, ECG and sweat-induced characteristics; third and fourth electrodes that concurrently provide (1) a signal source for the first and second electrodes for measurement of one or more of the physiological parameters and (2) a ground for the first and second electrodes; and a subject-securing device for securing the plurality of electrodes against the subject.

In accordance with another exemplary aspect of the disclosed subject matter, there is provided a physiological sensor system to be worn on a subject's skin to measure one or more physiological parameters, the system comprising: first and second electrodes to measure, when in contact with the subject's skin, physiological signals indicative of the physiological parameters; at least one third electrode disposed in contact with the subject's skin adjacent to one of the first and second electrodes configured to concurrently (i) measure impedance between one of the third electrodes and the corresponding first or second electrode, and (ii) produce a shielding signal that is in phase with and has a voltage equal to, less than, or greater than that of said physiological signals; and a subject-securing device for securing the first, second, and third electrodes against the subject's skin in operation.

In accordance with another exemplary aspect of the disclosed subject matter, there is provided a multi-parameter sensor system for measuring physiological signals in a subject, the physiological signals being indicative of physiological parameters, the physiological signals in certain embodiments being susceptible to interference with non-signal artifacts, the system comprising a plurality of electrodes configured to concurrently provide multiple functionalities, the multiple functionalities including in some embodiments, two or more of the following: measuring at least one physiological signal, reducing non-signal artifacts, providing a current source, providing a current sink, and providing a reference, the system comprising: at least one pair of electrodes that measure physiological signals indicative of physiological parameters; third and fourth electrodes that provide a reference signal for the sensing electrodes and, in some embodiments, also provides one or both of a ground and a reference; and at least one fourth electrode adjacent to one or more of the first and second electrodes that concurrently (i) measures the impedance between the fourth electrode and the adjacent electrode and (2) is configured to drive a shielding signal, wherein said shielding signal is in phase with the sensed signal of the respective sensing electrode; and a subject-securing device for securing the first, second, third and fourth electrodes against the subject's skin.

In accordance with another exemplary aspect of the disclosed subject matter, there is provided a physiological sensor system to be worn on a subject's skin to determine one or more physiological parameters, the system comprising: at least one pair of first electrodes that measure, when in contact with the subject's skin, physiological signals indicative of the physiological parameters; and a subject-securing device for securing said measurement system against the subject's skin, said subject-securing device comprising a deformable harness to be fitted around the subject's body, and one or more substantially rigid sensor mounting modules mechanically coupled to said harness via one or more couplers, wherein said harness, when fitted around the subject's body, applies an inward force to said one or more sensor mounting modules via said one or more couplers to urge the said sensing electrodes against the subject's skin to substantially secure a position of said sensors against the subject's skin.

In accordance with another exemplary aspect of the disclosed subject matter, there is provided a multi-parameter sensor system for measuring physiological signals in a subject, the physiological signals being indicative of physiological parameters and, in some embodiments, being susceptible to non-signal artifacts, the system comprising: at least one pair of first electrodes configured to measure physiological signals indicative of physiological parameters; a second electrode that provides a reference for the at least one pair of first electrodes; and a subject-securing device for securing the first and second electrodes against an outer surface of the subject; wherein at least one first electrode is affixed to a subject-facing side of a substantially rigid sensor mounting module to be immovably secured to a location on the outer surface of the subject by the subject-securing device.

### BRIEF DESCRIPTION OF THE FIGURES

Several embodiments of the present disclosure will be provided, by way of examples only, with reference to the appended drawings, wherein:

FIG. 1 is a schematic perspective view of an embodiment of the instantly disclosed subject matter;

FIG. 2 is a schematic bottom and associated cross-sectional view of one possible arrangement of a sensing electrode and conducting electrodes, including a thermal and acoustical sensors, in accordance with one embodiment of the instantly disclosed subject matter;

FIG. 3 is a schematic bottom and associated cross-sectional view of an exemplary arrangement of an electrode on a mounting assembly, in accordance with one embodiment of the instantly disclosed subject matter;

FIG. 4 is a schematic cross-sectional view of an exemplary arrangement of an electrode on a mounting assembly in use, in accordance with one embodiment of the instantly disclosed subject matter;

FIG. 5 is a schematic diagram of an exemplary circuit structure with shared use of conductive elements for measuring bioelectrical signals, in accordance with one embodiment of the instantly disclosed subject matter;

FIG. 6 is a schematic diagram of an exemplary circuit structure with shared use of conductive elements for measuring bioelectrical signals, in accordance with one embodiment of the instantly disclosed subject matter;

FIG. 7 is a schematic diagram of an exemplary sensing electrode with conductive driven shield electrode, along with a schematic representation of an accompanying circuit in an exemplary working relationship therewith, in accordance with one embodiment of the instantly disclosed subject matter;

FIG. 8 is a schematic diagram of one possible arrangement of a sensing electrode, conducting electrodes, and portions of a mounting assembly, along with a schematic representation of an accompanying circuit in an exemplary working relationship therewith, to include facility for sweat sensing, in accordance with one embodiment of the instantly disclosed subject matter;

FIG. 9 is a schematic cross-sectional view of a thermal sensing electrode in working relationship with a sensing electrode and mounting assembly, in accordance with one embodiment of the instantly disclosed subject matter;

FIG. 10 is a schematic diagram of an exemplary circuit structure with shared use of one electrode for concurrently measuring a plurality of bioelectrical signals representative of different physiological parameters, in accordance with one embodiment of the instantly disclosed subject matter;

FIG. 11 is a schematic diagram of an arrangement of measuring and reference electrodes with accompanying circuit structures in a working relationship, in accordance with one embodiment of the instantly disclosed subject matter;

FIG. 12 is a schematic diagram of a circuit structure that is configured to isolate non-signal artifacts, in accordance with one embodiment of the instantly disclosed subject matter;

FIG. 13 is a graphical representation of a relationship between non-signal artifact coupling and non-signal artifact frequency in the circuit structure of FIG. 12;

FIG. 14 is a schematic diagram of an arrangement of a subject securing device, a mounting assembly, and electrodes attached thereto, in accordance with one embodiment of the instantly disclosed subject matter;

FIG. 15 is a plan view of a mounting module with a plurality of electrodes attached thereto in accordance with another embodiment of the instantly disclosed subject matter;

FIG. 16 is a plan view of a mounting module with a plurality of electrodes attached thereto in accordance with yet another embodiment of the instantly disclosed subject matter;

FIG. 17 is a perspective view of an embodiment of the instantly disclosed subject matter; and

FIG. 18 is a graphical view of data output representations of ECG, respiration, and CO deflection data over time, in accordance with one embodiment of the instantly disclosed subject matter.

### DETAILED DESCRIPTION

In general, the disclosed subject matter relates to devices, systems and methods for determining one or more physiological parameters of a subject based on detected or measured physiological signals using a plurality of sensors and electrodes placed on or near the skin of the subject. Many of the plurality of sensors and electrodes are configured to provide more than one function, and in some cases, such functions are provided concurrently. Concurrently may mean that the functions are provided simultaneously, or that any of the functions are available at any given time (and can be switched therebetween as may be required), or that, at any given time, switching between the functions is of sufficient speed and/or time for provision of each function to provide the requirements of that function. As such, multiple physiological parameters can be assessed concurrently in real time and/or at any given time; additionally, the sensors or electrodes can operate to reduce interference (such as, but not limited to, non-signal artifacts from subject motion, non-signal electrical artifacts, salt-ion diffusion, static electricity, and capacitive or inductive artifacts), in some cases while concurrently providing one or more physiological parameters. As, in many cases, the physiological signals are of such low values relative to non-signal artifacts, many of the sensors or electrodes in some embodiments may concurrently provide both determining/measuring functions as well as functions relating to either the reduction of such non-signal artifacts or physiological signal detection despite non-signal artifacts. In addition, some embodiments provide for additional electrical components and/or circuit arrangements that assist in removing and/or accounting for non-signal artifacts during detection/measurement of physiological signals. As non-limiting illustrative examples, some signal buffers may be incorporated into circuitry to filter signals from specific frequency domains associated with specific physiological signals, some electrodes may divert and/or capture non-signal artifacts from measuring sensors or electrodes, some sensors or electrodes may generate a driven shield to prevent or reduce capacitive or inductive interference to measuring sensors or electrodes, and come circuit layouts may utilize differential noise filtering/isolation by splitting a signal between sensing and non-sensing electrodes (and utilizing the differential signal to remove non-signal artifacts from the sensed signal). The concurrent functionalities may also provide for reduced energy usage in some embodiments since some functionalities may be switched on or off at specific time intervals when a specific functionality, such as a measurement of a given physiological signal and/or related non-signal artifact reduction functionalities therefor may not be required at specific times. In some embodiments, the plurality of concurrent functionalities made possible by the various sensors and electrodes can be carried out utilizing a relatively small footprint that can secured to a subject with minimal discomfort and/or interference with activities or motions of such subject. Additionally, the layout and/or size and shape of the structures that are used to hold the sensors and/or electrodes in place against or near a subject minimize some non-signal artifacts; this may include, as a non-limiting illustrative examples, the securing device that holds sensors and/or electrodes against the subject so that motion between the subject and the sensor and/or electrodes is minimized, rigid or semi-rigid structures upon which sensors and/or electrodes are mounted having a shape that conforms to a subject's shape at the location where the sensors and/or electrodes contact the subject, rigid structures upon which sensors and/or electrodes are mounted having a geometry that extends beyond the sensors and/or electrodes on a subject-facing side of the rigid structures that maximize non-moving contact between such sensors and/or electrodes with the subject, and sizes and shapes of sensors and/or electrodes that minimize non-signal artifacts. In some embodiments, there is a centrally-located control module that facilitates the functionalities of each of the sensors and/or electrodes and/or circuitry and/or electrical components to provide concurrent functionality and minimize power consumption. In some such embodiments, the centrally-located control module is configured to store (in local memory) and/or transmit information related to the physiological signals or physiological parameters for use, storage and analysis to other transmitting and/or computing devices; such transmission may be provided by wired connection or non-wired connection known to persons skilled in the art (e.g. BlueTooth™).

The disclosed subject matter, therefore, according to an exemplary aspect, provides for a multi-parameter sensor system for measuring, processing and storing physiological signals in a subject. In some embodiments, the sensor system utilizes dry active and direct-surface contacting electrodes, which do not require, but can be used in association with, conducting creams or gels or other skin preparation techniques (such as abrasion or puncture). The physiological signals that are being measured by the one or more electrodes are indicative of physiological parameters, and due to their low magnitude relative to non-signal artifacts (i.e. noise), are susceptible to interference from such non-signal artifacts. The system, in one exemplary aspect, comprises a plurality of electrodes configured to provide multiple functionalities, in some cases concurrently, such functionalities including a measurement of one or more physiological signals, reducing of non-signal artifacts, providing a current source, providing a current sink, providing a reference, and combinations thereof. The system described in this embodiment further comprises: at least one pair of first electrodes that are for sensing physiological signals indicative of physiological parameters, the physiological parameters may include body bio-impedance (possibly in cases where second electrodes that are acting as reference electrodes concurrently provide a signal source by driving a signal across the body of the subject where the pair of first electrodes are located), ECG, skin-electrode impedance (possibly in cases where electrodes that act as reference electrodes are not providing a signal source), and sweat-induced bioelectrical signals (possibly when the pair of first electrodes are individually cooperating with third electrodes that operate as conducting electrodes); one or more pairs of second electrodes may concurrently provide (1) a signal source for the pair of first electrodes in their sensing of one or more of the physiological signals and (2) a reference for the pair of first electrodes; in addition, in some embodiments, the second electrodes may operate with any two or more combinations of other electrodes during measurement to provide body bio-impedance, skin-electrode bio-impedance, ECG including multi-lead, sweat sensors, acoustic sensors, light sensors, motion sensors, radiation sensors and thermal sensors; and a subject-securing device for securing the sensing electrodes and the reference electrodes against an outer surface of the subject.

In some embodiments, there is provided a sensor system for determining various physiological parameters based on the measurement of certain physiological parameters of a biological subject, which may or may not be a human. In some embodiments, there is a central management module, with which a plurality of electrodes and/or other electrical components are in electrical connection; these components are secured to a subject-securing device, such as a belt, which holds the electrodes in place against an outer surface of a subject, and depending on the connection between two or more of the available electrodes on the system to form a circuit, a portion of the circuit may comprise the subject itself, with or without a measured signal being driven therethrough. Some of the electrodes also provide or act in concert with signal sensing functionalities, and others provide other circuit structure functionalities, such as parasitic current sinks, ground, reference, signal source, current source, a driven shield (to shield from capacitive or inductive non-signal artifacts), differential noise filtering/isolation, and other functionalities. The central administration module is operative to configure the operation of the electrodes in order to provide many of these functionalities concurrently while the system is in operation and fastened, for example, around a body portion of a subject.

In some embodiments, there are provided systems comprising one or more sensing electrodes. In general, an electrode is an electrical conductor used to make contact with a nonmetallic part of a circuit; for the instantly disclosed subject matter, the non-metallic part of the circuit is the subject itself, although the system may be used to sense signals on other substrates. The sensing electrodes of the instantly disclosed systems and methods, may in general comprise a multi-parameter sensing electrode that integrates multiple separate sensing subsystems. Some sensing subsystems that are integrated in disclosed systems may share two common subject-contacting sensing electrodes; in some embodiments, such sensing electrodes are used to sense and measure physiological signals relating to, for example, such bioelectrical signals such as bio-impedance through the subject (which may utilize a separate signal current that is passed through the body of the subject at or near the sensing site by two other electrodes, in respect of which the sensing electrodes can sense characteristics of such signal to determine a body bio-impedance). In some embodiments, skin-electrode impedance is measured across the electrode to subject interface (which does not require a driven signal across the subject at the sensing location), and an ECG sensor. The ECG sensor, in general, may be a transthoracic (i.e. between different locations on the thorax or chest) interpretation of the electrical activity of the heart over a period of time, as detected by combinations of one or more electrodes attached to the surface of the skin and recorded by a device external to the electrodes, and in some cases, can be characterized as the sensing subsystem that detects and amplifies the tiny electrical changes on the skin that are caused when the heart muscle depolarizes during each heartbeat. The detection and measurement of the electrical changes on the skin may not always be required to be measured in a transthoracic fashion, and other locations on the subject body may be possible, including but not limited to the wrist, inner arm, fingers, neck, etc. In some embodiments, the body bio-impedance, skin-electrode bio-impedance, and ECG are concurrently measured by a pair of sensing electrodes, sometimes in cooperation with a pair of reference electrodes that provide a reference for the sensing electrodes and which, at times, also drive a signal current across the sensing region for body bio-impedance measurements. In general, this may be accomplished by splitting the output signal into different processing circuits; for example, a first such processing circuit is configured to filter and measure a signal for a first frequency domain in respect of which a first parameter may be characterized (e.g. ECG operates at a low frequency of around 0.1 Hz and as high as 150 Hz) and the second processing circuit is configured filter and measure a signal for a second frequency domain in respect of which a second parameter may be characterized (e.g. bio-impedance analysis, which operates at a different frequency band above about 1 KHz). The body bio-impedance and skin-electrode bio-impedance may be measured concurrently by switching on or off the driven signal across the reference electrodes. Additional physiological information of interest is often modulated on this or other signals, for example breathing effects the amplitude of ECG R-peak's as well the chest impedance and hence can be extracted by further signal processing of the acquired raw signals. In addition, any one of the sensing electrodes may operate to form a circuit with the conducting electrodes to form a sweat sensor, which can both determine the existence of sweat, as well as any sudden changes in the characteristics of sweat; both of these may be determined in some embodiments by measuring changes in in measured bio-impedance between a sensing electrode and a corresponding conducting electrode. Some such changes, based on a characteristic rate, magnitude or timing of such change, can be associated with the appearance of sweat on the surface of the skin between the sensing and conducting electrodes; and some such changes can be associated to changes in sweat characteristics based on other types of changes related to a physiological change in the subject relating to, for example, a change in health, activity, motion, or other status of the subject.

In some embodiments, there may be sensors and electrodes; in some cases, a sensor may be an electrode if such electrode is utilized for sensing bio-electrical signals which are used in determining physiological signals. In some cases, a sensor may not necessarily be an electrode since, for example, it may be utilized to measure other types of physiological signals, such as may be the case for, in some exemplary embodiments, light sensors and/or accelerometers and/or temperature sensors and/or acoustical sensors.

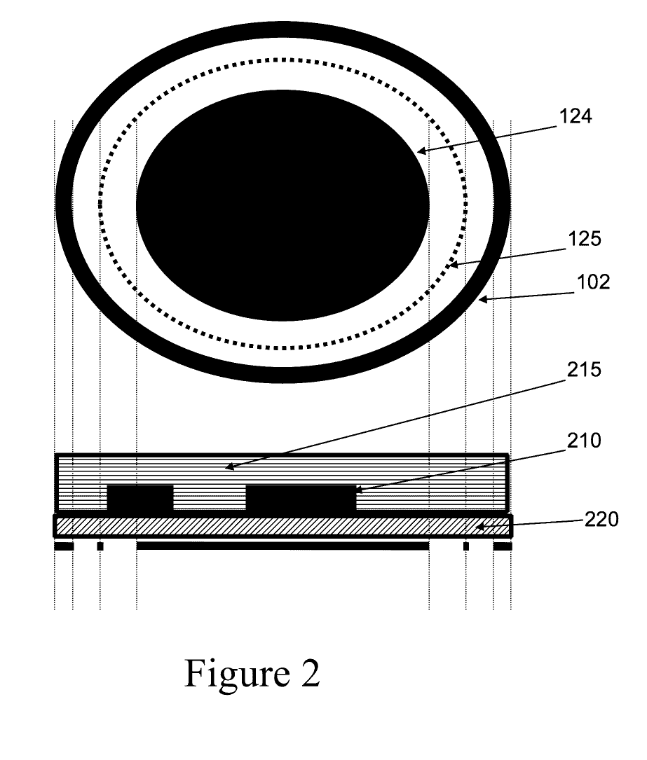

In some embodiments, there is provided a multi-parameter electrode that integrates multiple separate sensing subsystems and which shares common electrode-subject contact sensor components. With reference to FIG. 2, and in accordance with one embodiment, an example is provided of a sensing electrode and a conducting electrode, as well as complementary electrical and structural components. A sensing electrode 124 is centrally located as a circular electrode, in some embodiments so that the surface area of the sensing electrode is sufficient to capture low-level signals, while providing sufficient radial distance between the centre region and the edge of the sensing electrode, wherein edge effects that may cause non-signal electrical artifacts are minimized relative to the available area for collecting physiological signals. A conducting electrode 125 is situated around the sensing electrode. In the embodiment of FIG. 2, there is a slightly protruding buffering region ring 102 providing additional physical protection against motion-induced non-signal artifacts, as well as a containment mechanism for surface fluids (e.g. sweat). The buffering region ring is configured to improve the non-moving surface contact of the sensing electrode around which the buffering region ring surrounds; in some cases, the buffering region ring is also capable of drawing static or other electrical artifacts away from the edge of the sensing electrode, thereby reducing non-signal edge effects from the sensing electrode. While in the instantly described embodiment, a buffering region ring is used to describe the buffering region structure that substantially or partially circumscribes or surrounds the electrode, other geometric shapes for the buffering region structure may be used without departing from the scope and nature of the subject matter described herein; for example, square, rectangular, elliptical, polygonal, and lines along one or more sides of the electrode.

In some embodiments, the diameter of the sensing electrode is configured to be sufficiently large to both ensure that an area large enough for signal collection is maintained in non-moving (or motion-minimized) contact while maximizing the distance from such signal collection area from the edge of the sensing electrode (and/or buffering region edge). In some embodiments, the size of such sensing electrode is at least 0.5 cm; in other cases, it is at least 1.0 cm. Different sized sensing electrodes may be removably attached, as for example by removably attaching different types of mounting modules that have different sensing and other types of electrodes and sensors which have different sizes or characteristics, so that different sized electrodes that are most or more appropriate for the activity and/or subject can be switched onto the system; in this case most or more appropriate means that non-signal electrical artifacts are avoided or eliminated optimally, since some subjects and/or activities and/or locations can induce differing levels of non-signal electrical artifacts. The side elevation cross-sectional view in FIG. 2 depicts the solid mounting platform 220 upon which the electrodes 124, 125 are affixed, resulting in the mounting of the electrodes on the sensor mounting module 215. Also shown are the attached electrical components and connections 210, possibly including additional sensing apparatuses, such as an acoustical sensor or a thermal sensor (not shown). A multi-parameter electrode that integrates multiple separate sensing subsystems and sharing common electrode-body contact sensor component. As part of the proximally connected electrical components 210, there is provided in some embodiments a buffer amplifier located near the sensing electrode 124, and possibly a signal splitter, followed by one or both of an (i) ECG differential amplifier and gain block and (ii) a bio-impedance differential amplifier. These are specifically directed to the respective measurement of ECG and bio-impedance. By integrating the multiple sensors into a single sensing platform there may be provided, in some embodiments, coordinated measurement with various sensing subsystems thereby reducing or eliminating the interaction between the subsystems; reducing the physical space requirements for placing the multiple electrodes on the body; reducing the back end sensing electronics, cables and packaging materials (which in addition to reduced materials and cost savings, also reduced non-signal artifacts); and reducing power consumption requirements.

There are provided in some embodiments, conducting electrodes which may concurrently act as electrodes in a bioelectrical signal analysis circuit (e.g. in the detection and analysis of sweat), as a conductor for the generation of a driven shield (in which the conducting electrode, through the use of feedback circuit, possibly with amplifier, is driven with a current that has the same phase and, in some cases, the same or greater voltage). This signal is driven through the conducting electrode near the sensing electrode. This reduces capacitive effects on the sensing electrodes from surrounding structures, which would otherwise result in non-signal artifacts acting on the sensed signal. In some embodiments, the driven shield is restricted to the conducting electrode at the subject-facing surface, but it may be applied back through the sensor mounting module, and connected to circuitry to reduce any such capacitive effect all the way back to the central management module. In addition, the conductive electrode may be, with or without a resistor, connected to a ground, thereby drawing parasitic current away from the sensing electrode, which could otherwise generate non-signal artifacts.

In some embodiments, one or more of the electrodes which, either alone or in concert with other components, provide signal sensing functionalities are utilized in a manner to reduce or minimize the contribution of non-signal electrical artifacts from interfering with the signal. In some cases, this reduction or minimization results from the physical arrangement of the system, in some cases it results from the configuration of electrical components within the one or more circuits relating to the measurement of the physiological signals, in some cases it results from the analysis and management of the physiological signals themselves, and in some cases it results from a combination thereof. For example, some sensing electrodes are located on a subject-facing side of a rigid or substantially rigid mounting apparatus, which in some embodiments, also includes a buffer zone around the perimeter of the sensing electrode, which ensures a non-slipping and persistent contact with a flexible surface, whose movements relative to the surface of the subject may induce electrical signals or currents that would result in non-signal artifacts that the system would not be able to distinguish from one or more physiological signals. In some cases, the physical arrangement may also include a moveable and/or pivotable and/or articulable connection element that cooperates with a connection element on the subject-securing device to decouple motion on the subject-securing device and/or the subject, from motion on the mounting apparatus at the sensing site of the respective electrode on that mounting apparatus. Indeed, the flexible and/or elastic features of the subject-securing device also contribute to the decoupling of motion to the sensing site. In other cases, the physical shape, geometry, respective sensor or electrode feature ratios, and proximity to other sensors or components (both electrical and non-electrical) of the various electrodes also contribute to the ability to protect the signal from non-signal artifacts; these include the relatively (to the adjacent conducting electrode) large circular shape of the sensing electrode, which permits sufficient current between the skin of the subject and the sensing electrode while minimizing the effect of the edge effects of that electrode due to the distance between the edge and the central region thereof. In addition, the relative close proximity of the conducting electrode permits the attraction of non-signal and parasitic current by permitting a desirable route for such current when in proximity to the surrounding region and regions proximal to the measurement sensor edges. In addition, since the electrical connections of the multiple measurement sensors, conductive electrodes, and reference electrodes may be managed by the management module, it is possible to utilize differential signal measurements across different circuits or through different electrical components to distinguish between and/or isolate non-signal artifacts. It should be noted that different sizes of the respective electrodes may be used in some subjects that balance these competing objectives differently. For example, different sized sensing electrodes and reference electrodes may be used on subjects of small size (e.g. infants) than on subjects of much larger and different compositions (e.g. adults, large mammals such as farm animals).

In some embodiments, a sensed physiological signal can be an input that is split into signal sensing modules or subsystems that measure or analyze different aspects of the signal to be able to simultaneously determine multiple physiological parameters. For example, a signal may be split and utilizing buffer amplifiers and/or filters, an assessment of different aspects of the signal which exist in different frequency domains can be made, for example ECG, which signal operates at a relatively low frequency domain, and bio-impedance, which operates at a relatively high frequency domain, can each be measured in the respective splits of the signal. In cases where only one of these measurements is required, the split to the non-required measurement can be switched off to conserve power. In some embodiments, there may be a switch to divert the signal to the appropriate electrical component that is optimized for the desired measurement; in some cases, the switching can occur at a rate so that the measurements are perceived to be simultaneous. In the latter case, other aspects or electrode characteristics (or indeed the switching rate) may be adjusted or optimized in order to minimize the non-signal artifacts that may be generated by the switching. In some cases, where both of the physiological parameters may not be required, one or more electrical components may be switched off to reduce power consumption.

In some embodiments, the sensor system may comprise sensing electrodes wherein an output of at least one pair of sensing electrodes is split into a first and second buffer amplifier, the first buffer amplifier being optimized for ECG signal buffering and the second buffer amplifier optimized for bio-impedance signal buffering, the output of each being coupled to a switching circuit for selectively coupling either the ECG or bio-impedance output signal to pass through to a dual processing circuit which can be used to calculate either bio-impedance or ECG depending on which buffer the switch is connected to; in some cases, the dual processing circuit can selectively calculate the respective parameter by selectively assessing frequency bands associated with the frequency band associated with the respective parameter; for example, bio-impedance is measured from a higher frequency signal (˜1 KHz) and ECG signals are measured at lower frequencies (˜0.5 Hz to 150 Hz). In cases where measurements in the dual processing circuit consumes differential amounts of power depending on how many and which frequency bands are being assessed, the dual processing circuit may be configured to selectively process only a single frequency band, particularly with a preference for processing the frequency band that utilizes less power.

In other embodiments, a signal output of one or both of a pair of sensing electrodes may comprise an output that is an input to a single buffer amplifier optimized for both ECG and bio-impedance signal buffering, the output of the buffer amplifier being split into first and second processing circuits, the first processing circuit optimized for ECG signal processing, by filtering for the ECG frequency band at <−150 Hz, the second processing circuit optimized for bio-impedance signal processing by filtering for the bio-impedance frequency band at >−1 KHz.

In some embodiments, both electrodes of at least one pair of sensing electrodes are each coupled to a buffer in close proximity to the respective electrode, so as to minimize non-signal artifacts (e.g. parasitic currents), with the output of each buffer being split, a first split from each buffer being combined into a differential input of an ECG signal processing circuit, and a second split from each buffer being combined into a differential input of a bio-impedance signal processing circuit. The differential signal inputs into the respective signal processing circuits can provide for noise detection, measurement, isolation, and removal of such detected or isolated noise and/or other non-signal artifact from any calculations/measurements of the physiological signal.

In some embodiments, the use of distributed reference electrodes can be implemented. These may or may not be located on the mounting apparatus, and in some embodiments are woven directly in to the fabric on the subject-facing side of the subject-securing device. The reference electrodes can, in addition to providing a reference for sensing electrodes and/or sensors, simultaneously provide a distributed ground for some circuits (or bio-electrical signal measurement subsystem), and can provide current injection in another (or for the same circuit at different times). It should be noted, however, that other electrodes can provide a ground reference or reference signal for any other electrode/subsystem, and the use of the term reference electrodes may be used to refer to any such electrodes in the system. For some embodiments, the ground and reference electrodes may be the same, while in others, the ground electrode may be from a different electrode than from reference electrode and with different impedance values. Moreover, a sensing electrode and/or a conductive electrode in one operational configuration, may serve as a reference or ground electrode in another operational configuration at a different time, as determined by the control module. Since typical impedance for a ground electrode should be less than 500 kΩ, in order to reduce power line and/or static noise, some embodiments may be used to increase surface area of dry ground electrode and/or to wet skin with body lotion to provide a better noise dissipation. In embodiments, the woven conductive fabric, which is highly flexible, may be used on the subject facing side of the subject-securing device to increase the surface area of the ground electrode at all times on a region of contact. In some embodiments, a resistor is included in a circuit structure between a reference electrode and the signal ground, which results in current passing across or through the subject through to the other reference electrode and into its respective signal ground, thereby permitting the reference electrodes to concurrently act as both signal grounds, reference electrodes and a source of current.

In some embodiments, the first and second reference electrodes are electrically connected to a ground signal, wherein there is a resistor between one of the reference electrodes and a first ground signal and the second reference electrode is electrically connection directly to a second signal ground. This, in embodiments permits the pair of reference electrodes to concurrently dissipate static and other noise artifacts, as well as act as a ground reference when a BIA AC source current is injected into the body using the electrode with the contained resistor; the AC signal is isolated from the ground signal if the body impedance at the injected AC frequency is lower than the resistor impedance. The resistor may, in embodiments, be between 20 kΩ and 500 kΩ.

In some embodiments, there are one or more conductive electrodes which are located adjacent to corresponding sensing electrodes. Conductive electrodes may be, in some embodiments, located around an outer perimeter of sensing electrodes or portions thereof; in some embodiments, the conductive electrodes may form a complete or partial ring around the sensing electrode. This conductive electrode may provide one or more different functionalities in some embodiments. The surface of the conductive electrode may provide for a physical buffering region ring around the sensing electrode that provides for additional immovable contact at the sensing electrode by assisting to immobilize the sensing electrode relative to the surface of the subject; this physical buffering region ring around the sensing electrode (which may comprise of either an electrode, such as the conductive electrode, or a non-electrode physical structure) may minimize the effects of motion and pressure on the sensed signal at the sensing electrode by limiting deformation and pressure changes at the interior of the mounting apparatus, particularly when in cooperative use with the subject-securing device. The conductive electrode may also be driven with a signal that is in-phase with a sensed signal collected by an associated sensing electrode, in some embodiments using a feedback circuit structure with or without a voltage amplifier; the conductive electrode therefore reduces capacitive or inductive effects on the sensing electrode that may be caused by non-signal artifacts. The conductive electrode may operate as an electrode in a circuit with the corresponding sensing electrode; a measurement of bio-impedance therebetween, and in some cases a change in such bio-impedance of a particular magnitude, can be indicative of the existence of sweat, and moreover, further changes in such bio-impedance after the existence of sweat has been determined may further indicate a change in certain physiological conditions (e.g. aerobic vs. anaerobic exercise, shock, immobility, etc.) and/or the occurrence of an adverse health event (e.g. a stroke or heart attack, etc.).

In some embodiments, the subject-securing device is the component that holds many of the elements of the system together, as well as to urge the close and substantially immovable contact against the subject. In some embodiments, the subject-securing device may comprise a strap or other securable band that is removably placeable around a body portion of the subject. It may be a flexible, elastic device for placing around a portion of a subject, or it may comprise other mechanical means that forcibly clamp on to or squeeze against a portion of a subject. Alternatively, all or portions of the subject facing side of the subject securing device may comprise adhesive for being securely placed or urged against a subject. Combinations of these or other securing methodologies can be used without departing from the scope and nature of the instantly disclosed subject matter. In an exemplary embodiment, there is provided a flexible, elastic subject-securing device for securing the system to a subject, such as a chest belt. The coupling of the electrodes to the subject-securing device isolates the electrode-body interface from electrode-subject-securing device attachment, and minimizes effects such as stretching, pressure, limb movements, etc. of the subject-securing device which may otherwise cause the electrodes to move relative to the subject and thus cause non-signal artifacts from such motion. In embodiments, portions of the contact region of the subject-facing portion of the subject-securing device can be rendered conductive (e.g. by using woven electrically conductive fabric in the subject-securing device), and electrically connected to the central management module, and thereby can additionally provide means for electrical path between the electrode and other functions on the belt, such as separate pieces of electronics or other electrodes.

In embodiments, there is provided a coupler for attaching the sensing mounting apparatus to the subject-securing device; in some cases, the coupler is conductive in order to provide both a point of connection and maintain electrical connection of the one or more circuits including electrodes on that sensor mounting module. In embodiments where the coupler is articulable (including movable, pivotable, and rotatable) in order to decouple the sensor mounting module from the subject-securing device while maintaining an electrical connection, the coupler may be conductive in order to provide both a point of electrical contact and the coupling function. If conductive, the coupler may provide for one or more connections. In some cases, an alternative electrical connection can be used, which use an articulable coupler, such as a separate bendable or deformable wire. Couplers such as snaps, rivets, glue and thread made of conductive materials are examples conductive and non-conductive coupler types. Items such as Velcro, sticky tape, glue, thread are examples of other conductive or non-conductive means. The subject-securing device may in some embodiments provide protection or cushioning for the subject-facing side of the electrode from motion, pressure and temperature effects. Shielding materials could also be in the belt above the electrodes to provide shielding effects if required (e.g. from RF sources such as cell phones, wireless transmitters, microwave ovens, etc. that can contribute non-signal artifacts).

The subject-securing device may also in some embodiments provide a small contact region for fixing the sensor mounting module or other electrode thereto. The single point of contact allows the electrode position to be fixed in the horizontal and vertical directions on the body of the subject and for the application of vertical pressure through the securing device-electrode contact region and through the neighboring regions where the freely moving subject-securing device presses onto the backside region non-skin surface of the electrode. By making the contact region small and by having some adjustability in the connection, provided for example by a moveable coupler, such as a ball-and-socket type of snap, non-signal artifacts from unwanted belt motion can be isolated from the electrode. In addition, this allows the subject-securing device to stretch and bend and twist, providing for solid and comfortable attachment to the body with minimal transfer of the effects of subject-securing device motion into the attached electrodes or sensor mounting module, including (and in some cases particularly) the motion relative to the subject.

In some embodiments, some or all of the electrodes may be attached to a sensor mounting module. The sensor mounting module is a rigid, or substantially rigid mounting component wherein one or more electrodes are attached to a subject-facing side. The sensor mounting module is further configured to be attached to the subject-securing device such that the sensor mounting module is pushed, held or urged against the skin or outer surface of a subject's body or part thereof. The mounting module provides a rigid surface, upon which one or more sensors are affixed, that conforms to a sensing site of a subject (or causes a non-rigid surface, such as skin, to conform to the surface of the sensing site). As such, with the force or pressure against the subject provided by the subject-securing device, there is a location in the central region of the subject-facing surface that is substantially immovably secure against the sensing site, irrespective of movement along such surface or in the subject-securing device. Moreover, an increase in force along the interface of the mounting module and the surface of the subject will be mainly directed at the edges of the mounting module. In some embodiments, there is provided a buffering region between the outermost peripheries of one or more electrodes affixed to the sensor mounting module which further protects the electrode from motion effects relative to the contacting surface. A solid (e.g. PCB, metal, plastic, silicon, glass) or semi solid (e.g. mylar, plastic, rubber, coated paper) material that provides direct contact to skin can form the bulk material of the sensor mounting module. The size, shape, materials can be of a wide range, but in general the size of the platform will be greater than or equal to the minimum sensor surface area. In the case of ECG and bio-impedance sensing in the chest region of an adult human this would be in the order 2 cm² or larger. The back side (non-skin contact side) or interior of the sensor mounting module can be used to house sensor electronics, mechanical, electrical and RF isolation mechanisms, buffers, subject-securing device attachment, other sensors and sensor-related devices (e.g. acoustical, light source and light sensors), heating elements, and electrical interconnect mechanisms. The sensor mounting module can provide solid, stable electrical, thermal and mechanical contact to skin surface and an area on which electronics can be mounted on non-skin side of platform. It may also help to reduce skin deformation effects such as voltage, current and thermal gradients caused by movement at the electrode-body boundary (for example due to motion artifact, pressure). The sensor mounting module may also provide in some embodiments more uniform electrical and thermal patterns on the entire surface of the electrode, as well as for integration of one or more sensing types and features on the rigid platform, and reduces the need for gels, moisture, skin abrasion or micro-needles to stabilize electrode-body contact effects.

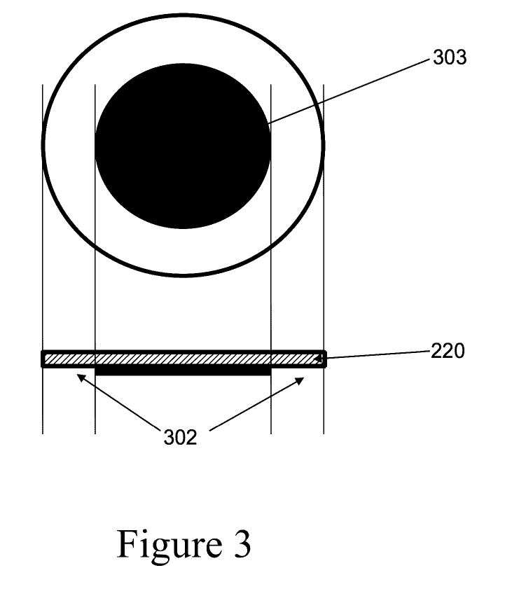

The sensing module may also provide an electrode deformation buffering region around each electrode. A solid or semi-solid regions formed by the sensor mounting module is provided on some embodiments surrounding one or more electrodes. With reference to FIG. 3, there is shown is an example of a mechanical buffer around the sensing electrode. A solid platform 220 with mechanical buffer zone 302 around the corresponding electrode (or electrodes) 303. Although FIG. 3 shows the sensing electrode as projecting slightly away from the surface of the mechanical buffer; in some embodiments, the sensing electrodes are coplanar or substantially coplanar with the mechanical buffer zone. This may be accomplished in some embodiments by overmolding the mechanical buffer zone until the material is flush, or substantially flush, with the mechanical buffer zone. Further, the edge of the mechanical buffer zone is shown in FIG. 3 as being substantially at right angle to the subject facing side; in some embodiments, this edge is significantly rounded, which, in addition to increasing the comfort for the wearer of the sensor, also reduces the sensor platform-to-skin motion at the edges of the mechanical buffer zone in order to reduce even further the sensor to skin relative motion, in turn reducing any electrical non-signal artifacts at the sensing electrode.

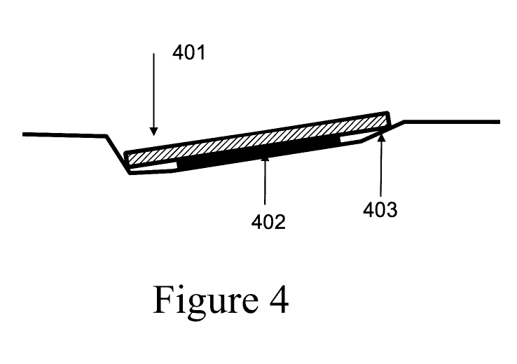

With reference now to FIG. 4, the configuration shown minimizes effects of motion and pressure 401 on the sensed signal by limiting the deformation in the region of the sensing element 402 and restricting the bulk of the deformations and pressure changes to the buffer zone 403 or outer edges thereof. The buffering region, formed by the extension of the rigid surface of the sensing mounting module around the periphery of electrodes, can be a wide variety of sizes, shapes, materials depending of the level of electrical, thermal or other form of isolation required. For electrical deformation effects caused by effects such as motion and pressure this regions should provide at least 1 mm and ideally in the range of at least 4-6 mm border around then entire sensing region. The buffering region provides in some embodiments an isolation region between edge of electrode and sensing region in contact with body such that effects of motion, pressure, and/or temperature are isolated from the contained sensing regions. The buffering region, in some embodiments, may also isolate deformation effects such as voltage, current and thermal effects to the buffer region, maintaining more stable and representative values in the contained sensing regions for the one or more electrodes affixed to the central region inside the buffering region.

In some embodiments, a sensor mounting module may comprise of additional sensors, such as an acoustic sensor, a light source with light sensor, a motion sensor such as an accelerometer and a thermal sensor. The acoustic sensor may, in embodiments, comprise a flexible diaphragm as input window of microphone imbedded into body of sensor mounting module, having a frequency band of approximately 20 to 1000 Hz; the acoustical sensor can thus measure or detect pulse, breathing rate, heart rate, or any other activity in the subject that creates or results in noise. Some acoustic sensors may comprise a piezoelectric microphone. The light source (e.g. a light emitting diode) emits light at one or more wavelengths and reflected off the adjacent skin, and depending on the absorption of particular wavelengths, as detected by the light sensor (e.g. photodetector, photodiode, or other light sensor known to a person skilled in the art), certain characteristics of the blood can be determined, such as pulse oximetry and SpO₂ (oxygen saturation).

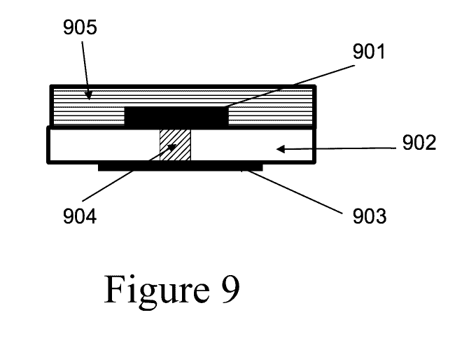

In embodiments with a thermal sensor, there are provided configurations with thermal sensors place in close proximity to the back side of an electrode that may be used for another purpose (e.g. a sensing electrode, a conductor electrode, or reference electrode, or other), wherein the sensing electrode is made of material that is both highly electrically and thermally conductive. In some embodiments, where additional thermal conductivity is desired, the electrode may have introduced therein as one or more plugs extending from the subject-facing side to the back side of such electrode, highly thermally conductive material, which will assist in ensuring that as an accurate measurement as possible can be made. With reference to FIG. 9, there is shown is an example of a thermal sensing element 901 mounted on a thermally insulating rigid platform 902. A thermally conductive plate 903 is in contact with the body and is connected to the sensing element 901 by thermally conductive plug 904 in rigid platform 902. Although not shown in FIG. 9, there may be a plurality of conductive plugs that extend through a sensor/electrode and or the rigid platform to facilitate accurate temperature measurement for the site of the sensor/electrode. Thermal insulating material 905 is placed over thermal sensing element 901 to isolate it from ambient temperature changes. This arrangement provides for simultaneous measurement of temperature with the other electrode subsystems and sharing of sensing elements in contact with the measured body. In some embodiments, rigid platform 902 may in fact be a sensing or other type of electrode or sensor. In such embodiments, there may nor may not be a requirement for the thermally conductive plate 903, although this may be integrally formed along the subject facing side of the corresponding other electrode in order to facilitate heat transfer through the electrode to the thermal sensing element 901. The thermal sensing element 901 may comprise of a number of different types of thermal sensors, such as a silicon bandgap temperature sensor, a base-emitter diode of a bipolar junction transistor, thermistors, thermocouples, IR detectors, or any other temperature sensing devices known in the art. The thermal insulating material 905 may be in some embodiments the bulk material of the sensor mounting module. In addition to providing a physiological parameter (i.e. body temperature), the capability to measure temperature may also improve measurement capabilities of the system for other bioelectrical signals; this includes at least the following two benefits: (1) since temperature may cause changes in certain bioelectrical signals that are unrelated to the underlying biological process or physiological parameter, the use of the current body temperature may be used in calculations to correct such signals or measurements from such temperature effects; and (2) some sensor mounting modules may include a heat source to maintain a constant temperature at a sensing site and temperature measurement can ensure a constant temperature and/or control over the heating mechanism. In addition, ambient temperature effects can be reduced with the use of temperature insulated deformation buffer region, as well as with thermal insulation provided by the belt covering the top of the electrode to augment the thermal insulation provided on top of the temperature sensing component in the electrode part. In embodiments, there may be provided an air-insulating dome, or other enclosing structure or material that provides thermal insulation from thermal noise sources, over the entire temperature sensing subsystem and/or thermally insulating overmolding material. One or more sensing elements can be used in one or more other electrodes to provide temperature readings at multiple points on the body surface. These readings can be used independently or in aggregation as required by any specific application. In some embodiments, a common sensing circuitry is provided for driving current and sensing voltage through the IR sensing subsystem.

In accordance with another exemplary aspect of the disclosed subject matter, there is provided a method of concurrently measuring two or more physiological parameters from a subject using a same pair of sensing electrodes and an associated pair of grounding reference electrodes, each in contact with the subject's skin to define a measurement site therebetween, the method comprising: collecting physiological signals via said pair of sensing electrodes; splitting said collected physiological signals in accordance with distinct frequency domains respectively associated with each of the distinct physiological parameters; and switching the reference electrodes between an active mode in which a current is provided across the measurement site and a passive mode in which said current is not provided, wherein said active mode and said passive mode cause the sensing electrodes to assess respective ones of said distinct physiological parameters. By switching from the active mode, in which a signal is driven across the location of the pair of sensing electrodes, to a passive mode, in which the driven signal is not present, the sensing electrodes can concurrently provide body and skin-electrode bio-impedance. Although in some embodiments this may not be strictly simultaneous at all times, each of the respective parameters can be measured at any given time by cycling quickly between these modes, and depending on the length of the cycle between active and passive modes, the measured results can be provided quasi-simultaneously since they are measured successively with a reduced time interval between such measurements, in some cases in alternating fashion.

In some embodiments, in addition to methods that provide for concurrent measurement functionalities, by virtue of the reference electrodes acting as a driven signal concurrently while providing a reference signal and/or a ground signal, the conductive electrodes can also provide concurrent functionalities. Such methods will provide for concurrent use of the conductive electrode in providing multiple functionalities: a driven shield, a measurement electrode along with the corresponding sensing electrode to act as a sweat sensor, and a current sink for parasitic current and/or noise during operation of the sensing electrode. In some embodiments, the conducting electrode is switched between modes to provide concurrent functionalities; in other embodiments, two or more of these functionalities may be provided simultaneously. In some embodiments, concurrent use of the sensing electrodes to measure both bio-impedance (including either or both body and skin-electrode types) and ECG is provided; either by a step of switching an output signal between buffer amplifier components which are each optimized for each parameter and then passing the outputs thereof to a respective signal processing unit appropriate for the frequency band of the parameter being measured. In some embodiments, the signal is split and therefore there is possibly simultaneous measurement of different parameters in different signal processing units.

There are provided in the instantly disclosed subject matter systems comprising electrodes, each of which concurrently provides two or more functionalities. The functionalities may include: concurrent measurement of multiple parameters; concurrent measurement of one or more parameters with the provision of one or more of a signal current, current sink or source, a ground signal, a reference signal, and a driven shield; or the concurrent provision of two or more of a signal current, current sink or source, a ground signal, a reference signal, and a driven shield. In some embodiments, any one or more of the sensing electrodes, the reference electrodes, or the conductive electrodes, or a combination thereof may provide concurrent functionality. As such, in some embodiments, there is provided a physiological sensor system to be worn on a subject's skin to measure one or more physiological parameters, the system comprising at least one pair of sensing electrodes that measure, when in contact with the subject's skin, physiological signals indicative of the one or more physiological parameters, the output of one sensing electrode of each pair being split into first and second processing circuits, the first processing circuit optimized for ECG signal processing, the second processing circuit optimized for bio-impedance signal processing, and a subject-securing device for securing said sensing electrodes and said first and second reference electrodes against the subject's skin in operation. The other electrodes (e.g. reference electrodes and conducting electrodes), if they are present at all, need not provide multiple concurrent functionalities in this embodiment. In embodiments, the first and second reference electrodes may provide only a source signal for said sensing electrodes in their measurement of the physiological parameters; they may only provide a reference signal, or a ground signal; and in some embodiments, they may provide all or some combination of these functionalities concurrently (along with the concurrent measurement functionalities at the sensing electrodes). In some embodiments, some electrodes may provide a sensing function and concurrently provide functionality as a ground, reference, or driven shield. In some embodiments, reference electrodes may act as noise (i.e. non-signal artifacts) attenuators that remove parasitic current; in some such cases, the reference sensors may act by drawing non-signal artifacts away from sensing electrodes (and their respective circuits).

As used herein, the term “concurrent”, with respect to two or measurements or modes, may mean simultaneous (i.e. the measurements or modes are made at the same or overlapping times) or it may mean the measurements are taken, or the modes are in existence, at different discrete intervals that are sufficiently close enough in time within a specific time period such that measurements or modes can be deemed to the same as if the measurements were taken or the modes existed at the same time. The latter may be associated with a rapid and repeated switching between measurements or modes, but it need not be repeated. In some cases, concurrent may refer to the ability to be in, or to take, any one or more of a plurality of modes, or measurements respectively, at any given time.

Some physiological signals may be determined by detecting and/or measuring any of a wide number of bioelectrical parameters known to persons skilled in the art, including voltage, current, frequency, resistance, impedance, reactance, capacitance, inductance, phase angle, or any other electrical characteristics which can be measured, calculated and estimated in respect of an electrical current or signal. In other cases, the physiological signals may refer to light, acceleration, noise, temperature, or other parameters relating to a subject that may, for example, be measured by sensors included in embodiments hereof.

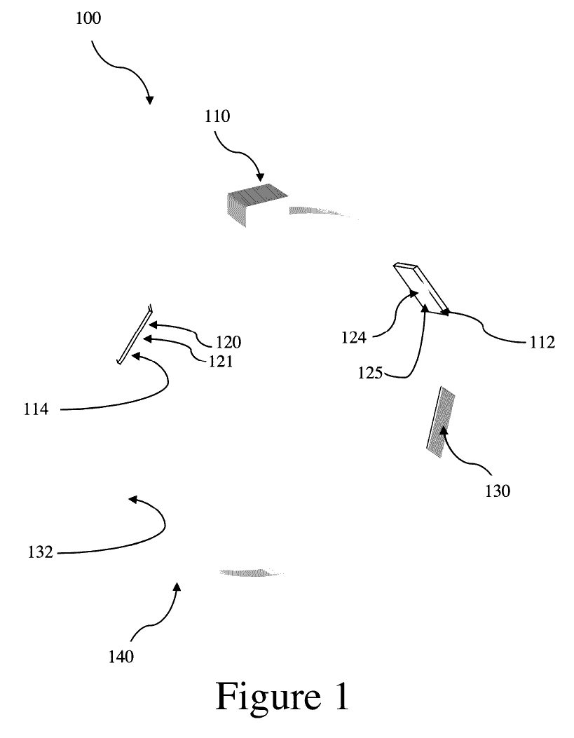

With reference to FIG. 1, shown is an exemplary embodiment of the subject matter disclosed herein. A sensor system 100 for concurrently measuring a plurality of bioelectrical signals in a subject, the sensor system 100 comprising a subject securing device 140 (the band) and a control module 110, which may or may not be contained in shown sensor mounting modules or in separate modules not shown, electrically connected to a plurality of electrodes 132, 121, 120, 124, 125, 130 (optical, temperature, sweat, accelerometer, radiation, or other electrodes not shown). There are two pairs of electrodes 120, 121 and 124, 125, each of which are secured on one of two substantially inflexible mounting modules 112, 114, and which mounting means are pivotally secured (pivotal mounting means not shown) to a flexible portion of the subject securing device 140. Each pair on the two mounting modules comprise a first sensing electrode 120, 124 and a shield conductor electrode 121, 125 therearound. There are also provided two reference electrodes 130, 132, which in this embodiment are flexibly and integrally affixed to a subject-facing side of the subject securing device 140 through a woven, stitched, laminating or other affixing process. The reference electrodes 130, 132 are located on the subject-securing device 140 such that in cases where they provide a source of current across one another, the sensing electrodes 120, 124 are located in between the reference electrodes 130, 132 albeit in some cases at an offset from the direct line therebetween due to the curvature or shape of the subject.

Each of the electrodes 132, 121, 120, 124, 125, 130 (as well as the optical and temperature and other possible electrodes that are not shown) are electrically coupled to the control module 110. The control module 110 can configure the connections between the electrodes to form bi-polar and multi-polar circuits using any combination of two or more electrodes. In addition, any of the electrodes can be used to concurrently do two or more of the following: act as a current source, act as a current sink, perform measurement, or signal protection/non-signal artifact reduction activities.

The shield conductor electrodes 121, 125 may, operate as either measuring sensors or as conductors for providing a driven shield around each of the first sensing electrodes. In embodiments, the circuit formed by the first sensing electrode 120 and the shield conductor electrode 121 measures certain electrical characteristics in the space therebetween and can thus be used as a sweat sensor. Changes in the electrical characteristics in that circuit can indicate the existence of sweat between the sensors, or changes in characteristics of the sweat that may be indicative of certain physiological changes in the subject.

In embodiments, one of the first measurement sensors 120 to other first measurement sensor 124, there is provided a circuit (a portion of which comprises the subject (not shown)) which includes circuit elements for providing ECG and bio-impedance measurement (not shown). In a four sensor configuration, the system 100 uses the reference electrodes 130, 132 to supply current across the measurement sight during the measurement of body bio-impedance across the two first sensing electrodes 120, 124. In some cases, there is a two sensor bio-impedance measurement, which is used to determined skin impedance or in some cases bio-impedance at or near the surface of the subject. In either case, and for ECG measurement, sweat measurement, as well as other measurements involving any of the other sensors, the outer electrodes also act as reference electrodes and/or ground. In some embodiments, the reference electrodes can provide a source of current from reference electrode 130 to reference electrode 132. In some cases, the reference electrodes can provide a source of current, but can still provide a signal ground for any one or more of the possible circuits by placing a resistor in one reference that is electrically connect to a current source, such that current will go to the signal ground at that reference sensor but also, due to the resistor, pass through the subject to ground at the other reference sensor. The configuration shown in FIG. 1 is exemplary and different configurations are possible; for example, control module 110 could be a separate element, or combined with one of the other mounting devices such as with mounting modules 112, 114 or distributed between the 2 (or more) electrodes.

In some embodiments, this dual capability of acting as both ground and current source for bio-impedance measurement is provided for by having a resistor on one reference electrode that is “near” or closer to a current source, which ensures that the current flows through measurement site rather than sinking straight to ground at the “near” electrode and can also sink to ground at the other reference electrode, and such current then provides a source signal for bio-impedance measurements between such reference electrodes.

The shield conductor electrode surrounds the primary electrode and is driven at same voltage phase to protect the signal in primary, but also may operate in a second mode where impedance is measured thereacross to measure the existence of sweat and also the change conditions of the sweat therein. In some embodiments, the surrounding conductor electrode may also be connected to ground in order to draw away non-signal parasitic electrical currents from the nearby primary electrode.

The sensor mounting modules 112, 114 or the subject-securing device 140 may also contain an optical sensor, which may comprise a light source, and a light sensing electrode, which in some embodiment may be a photodiode. In some embodiments, there is provided a temperature electrode, which in some embodiments may be a thermocouple placed on the opposing side of each of the first sensing electrodes 120, 124; in embodiments with a temperature electrode, there may be provide temperature conducting elements that provide high temperature conductivity through the first sensing electrodes 120, 124.

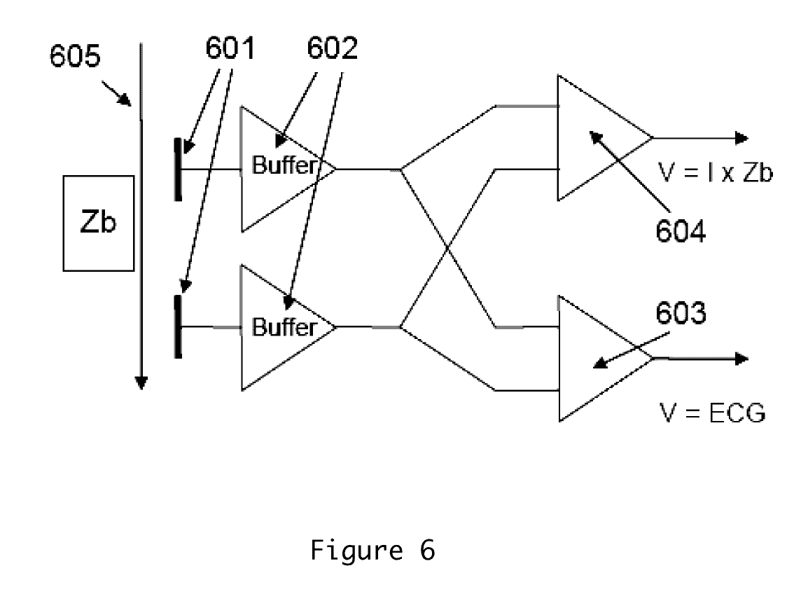

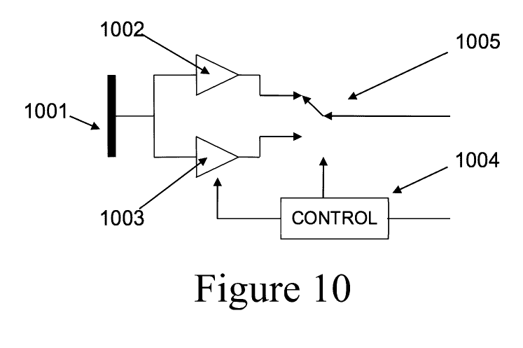

Some embodiments may be characterized as a system of electrodes for measuring physiological signals across a living organism, the physiological signals being indicative of a plurality of bio-electricity-based characteristics, the electrodes being specifically configured to simultaneously measure a plurality of such electrical signals, some of such physiological signal having very low electrical signals (potential and current), while eliminating, filtering, measuring, or otherwise accounting for any contribution of such signals from other sources during measurement. This capability to simultaneously measure a plurality of reduced strength signals, while accounting for the artifact signal, may be based on a number of functional aspects, some of which may include but are not limited to the following (wherein such list is not intended to be a comprehensive enumeration of aspects, requirements, or objectives, as these aspects may not be present in all cases and other aspects may be possible):
1. Rigid platform to maximize spatial and temporal contact to highly deformable surface, and thus minimize generation of galvanic skin response due to relative motion between the surface and the one or more electrodes and/or sensors
2. Respective size and ratio of physical buffer zone around sensing elements
3. Flexible and/or adhesive securing means to ensure firm and persistent contact to highly deformable surface
4. Shared used of different electrodes to form different circuits having any two or more the electrodes and/or connection to different electrical functional units;
   a. Differential signal noise interference measurement (see, for example, FIG. 6)
   b. Switching to separate buffers each optimized to provide specific gains associated with a specific type of signal measurement (see, for example, FIG. 10)
5. Coordination of signals across a combination of multiple electrodes and multiple electrical signal transformers that allows sensors to simultaneously measure multiple physiological signals from a single or low number of sites, while accounting for the low-level signal levels of such site where relatively higher non-signal artifacts may be present or generated; including a combination of some or all of the following:
   a. Independent optimization of signals and power management, through switching (time interleaving) or frequency band (multiplexing);
   b. Differential buffering to reduce signal noise
   c. Driven shield structures to reduce signal noise
   d. Frequency multiplexing to produce multiple measurements of different parameters from the same output of one electrode
   e. Simultaneous measurements to observe effects of one sensor on another
6. Introduction of higher thermally-conductive and thermally insulating materials into electrodes to minimize external temperature effects and maximize those of the skin and internal body temperature

A driven shield may in some embodiments, seek to match a signal between the sensing electrode and another adjacent component, thus making the parasitic capacitance being virtually nonexistent. A driven shield may be positioned roughly around the sides of the electrode and fed with a signal that is in phase with the signal that is sensed at the electrode. Since the shield and the electrode voltages are in-phase, capacitance between the two, as well as the electrode and all components positioned behind the shield, have reduced effect on the operation of the sensor.

With reference now to FIG. 3, there is shown is an example of a mechanical buffer around the sensing electrode. A solid platform 220 with mechanical buffer zone 302 around the sensitive element 303. This buffer zone may be flat with a right angle edge as shown or it may be rounded or other shapes that provide the mechanical buffering but cause less indentation to the subject surface (e.g. skin) at the electrode edge and thus be possibly more comfortable to the wearer while still reducing indentation or deflection of the subject (e.g. at the skin) at the electrode (or other sensor).

With reference now to FIG. 4, there is shown is an example of skin deformation. This configuration minimizes effects of motion and pressure 401 on the sensed signal by limiting the deformation in the region of the sensing element 402 and restricting the bulk of the deformations and pressure changes to the buffer zone 403.

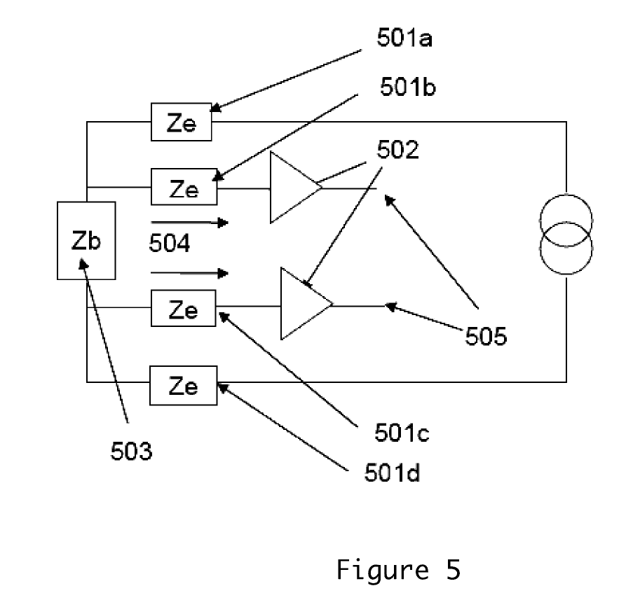

With reference now to FIG. 5, there is shown is an example of a tetrapolar bio-impedance sensing system using active dry electrodes 501a, 501b, 501c and 501d. The current driving electrodes 501a, 501d correspond to the reference electrodes that are woven into the fabric of the subject-securing device and which provide a signal, Zb, across a measurement site. The sensing buffer amplifiers 502 are placed in close proximity to the measurement site 503. This configuration of close proximity minimizes the parasitic effects that would provide a path for current flow 504 into the input of the buffer 502. Keeping current 504 small ensures that the detected voltage 505 accurately reflects the sample voltage 503. The circuit shown in FIG. 5 may be used for measurement of bio-impedance at a measurement site using 4 dry electrodes. The 2 outer electrodes 501a, 501d allow for current to flow to the 2 inner electrodes 501b, 501c. Current flows into the 2 inner buffers 502 and provides a differential output signal that represents the sample voltage. The buffers may be close to the measurement site to reduce noise. This circuit uses a simple and effective 4-pole configuration to produce the current through the measurement site and collect the sample voltage representative of bio-impedance after the buffers. In embodiments, a 4-wire approach may be used. Each of the buffer amplifiers 502 may, in some embodiments, comprise of sub-buffers (not shown). In embodiments, 501a and 501d can support 2-wire measurements in some configurations.

With reference to FIG. 6 there is shown is an example of an active electrode with shared use of conductive elements 601 and buffers 602 for ECG 603 and bio-impedance 604 voltage sense. There are two conductive elements (e.g., electrodes) 601 that facilitate a differential input current to the circuit, where each input line first goes through a buffer stage to buffer 602 from loading of the circuitry. The buffered differential signal is then split and passed through a bio-impedance differential amplifier 604 and an ECG differential amplifier (and gain block) 603. This allows for sensing of multiple signal types (e.g., bio-impedance and ECG) differentially but concurrently, and producing respective multiple single-ended outputs. The sensing system uses 2 electrodes to differentially input, while buffering each input, and sample multiple signal types from the differential input to create the individual single-ended outputs. This configuration may have robustness against noise using differential signaling with 2 electrodes and allows for multiple different signal types to be sampled and output to the rest of the system simultaneously.

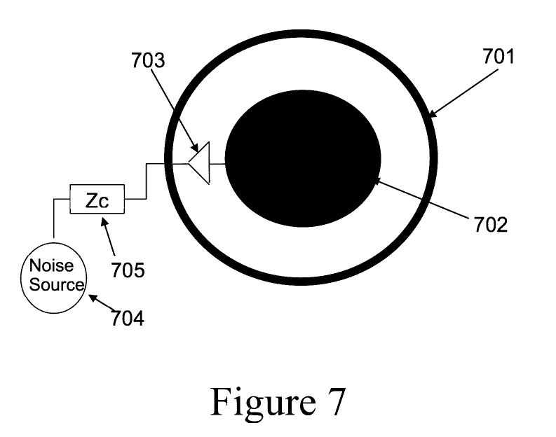

With reference to FIG. 7, there is shown an example of electrodes comprising a protective conducting driven shield electrode 701 around the central sensing electrode 702, with the conducting electrode 701 driven by a buffer amplifier 703 to be at the same potential as the sensing electrode 702. Noise sources 704 with parasitic coupling 705 to the electrode find a low impedance path to ground via the buffer 703 output and are therefore shunted away from the sensing electrode 702, thereby reducing the effect of noise source 704.

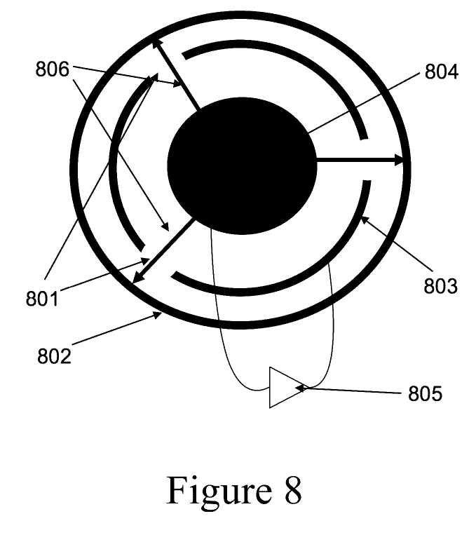

With reference now to FIG. 8, there is shown is an example of an electrode with sweat detection by surface moisture detection in microchannels 801. The outer ring 802 is connected to a known potential eg GND. The inner ring 803 is perforated with microchannels 801. The inner ring is driven by a unity gain buffer 805 to act as a driven shield around the central conductor 804. An AC impedance measurement is made between the central disc 804 and the outer ring 802. Impedance measured is dictated by current flow 806 through the microchannels 801 that contain surface moisture. The inner ring potential traps the current flowing through the body whereas the inner channel rings provide current flow that is correlated to various aspects of the surface fluids present. Measurements made at various frequencies provide increased sensitivity and accuracy as well as provide facility for ascertaining sweat composition, volume. When analyzed over time allow changes and trends of these parameters to be determined; such trends may be used to detect or indicate changes in the health or status of the subject, as well as general changes in physiological processes. Measurements taken alternating the inner ring potential provide a means of further improving the sensitivity and accuracy of the readings.

Integrating the multiple sensors into a single sensing platform offers many advantages including allowing for coordinated measurement with various sensing subsystems thereby reducing or eliminating the interaction between the subsystems; reducing the physical space requirements for placing the multiple electrodes on the body; reducing the back end sensing electronics, and cables and packaging materials. As such, in addition to providing multi-parameter assessment and non-signal artifact reduction, the overall footprint of the device is minimized and power reduction methodologies can be implemented when measuring some or all parameters.

With reference to FIG. 9, there is shown is an example of a thermal sensing element 901 mounted on a thermally insulating rigid platform 902. A thermally conductive plate 903 is in contact with the body and is connected to the sensing element 901 by thermally conductive plug 904 in rigid platform 902. Thermal insulating material 905 is placed over thermal sensing element 901 to isolate it from ambient temperature changes. Allows for simultaneous measurement of temperature with the other electrode subsystems and sharing of sensing elements in contact with the measured body. Other embodiments may have a dome located over the thermal sensor so as to provide an air insulation barrier over the entire thermal sensing region, thereby minimizing conduction, evaporative and other thermal effects from non-subject/surface sources. Embodiments may have radiated heat blocking material over top of the thermal sensor (e.g. to the belt that holds the electrode to the body) to reduce or help compensate for thermal effects.

With reference to FIG. 10 shows and example electrode assembly characterized by different electrical power and signal bandwidth regimes, allowing for independent optimization of signal and power management aspects of each measurement subsystem long term ECG operation and high quality bio-impedance measurements. The sensing electrode 1001 is connected to 2 (or more) buffers 1002, 1003. Each buffer can be optimized to deliver differing gains, frequency bandwidth response, multiplexing, amplifying, and powering as required for each subsystem, thereby allowing for the independent optimization of SNR, power utilization, frequency characteristics and product cost. For example, ECG buffer 1002 only requires a low bandwidth buffer sufficient for ECG measurements and may utilize an ultra-low power (i.e. nA) buffer amplifier. Another buffer 1003 is connected in parallel with the ECG buffer 1002 and is enabled by a control mechanism 1004. This second buffer is used for bio-impedance measurements whilst still allowing concurrent ECG measurements. A means of selecting which buffer drives the line 1005 can also be included to allow for sharing of signal wires from the electrode. The same or a separate means can be provided of enabling and disabling the separate buffer subsystems so that subsystems can be switched off to save power when not being used. A control mechanism 1004, may be present, to control the buffer powering and/or signal selection allowing for multiplexed or simultaneous measurement of the sensor subsystems. Allows for SNR/power savings tradeoffs. The circuit shown in FIG. 10 may also allow for measurement of multiple signal types (e.g., bio-impedance and ECG) in a time-interleaved manner. The single conductive element (e.g., electrode) 1001 inputs a signal current to the multitude of buffers 1002, 1003, each designed/tuned/optimized for a particular signal type. Examples of tuning parameters are power, bandwidth, amplifier gain, etc. At this point there are multiple signal type outputs: The circuit can then include a time-interleaving switch 1005 that is controlled to switch between the multiple of outputs, such that one of the outputs is selected to pass through to the single final output at a time. In alternative to the circuit in FIG. 10, a configuration without the switching element 1005 is supported: instead, the outputs are combined directly and the buffers are individually tuned for different signal types, such that the outputted different signal types are each at different frequency bands, and the combined output can be filtered and measured within respective frequency bands that are each associated with the desired measured parameter; as such, the parameters may be provided concurrently without utilizing a switching mechanism. The circuit in FIG. 10, for example, has the capability to sense multiple different signal types from a single measurement site in a simple manner such as these 2 configurations.

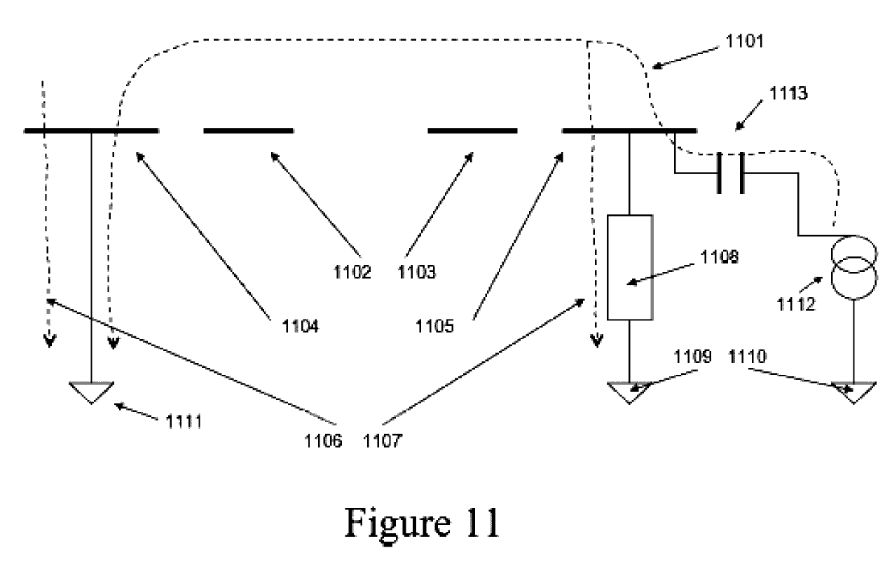

With reference to FIG. 11, there is shown a schematic and diagrammatic representation of the bio-impedance signal and sensing paths. Sensing electrodes 1102, 1103 are used to measure bio-impedance and ECG, in accordance with embodiments of the instantly disclosed subject matter. The reference electrodes 1104, 1105 are configured to provide a signal path for bio-impedance signal current 1101, but it also concurrently acts as ECG ground current 1106, 1107 and can be used as signal measurement points (2-wire bio-impedance). The current signal generator 1112 generates current 1101 which is directed over capacitor 1113, and due to the resistor 1108, some or all of the signal current path passes through the body of the subject, rather than directly to ground via 1109, and as such also goes to ground at 1111.

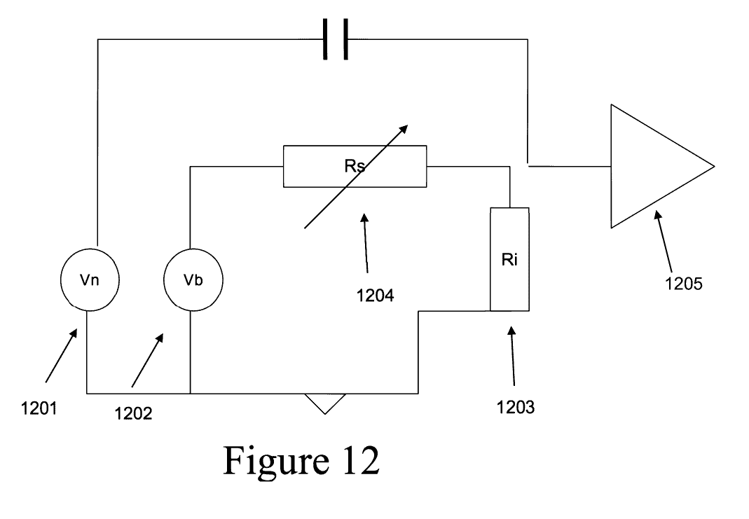

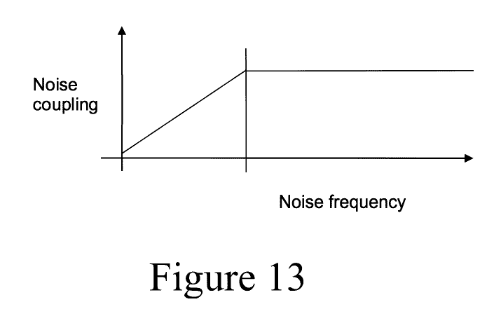

With reference now to FIGS. 12 and 13, there is provided in FIG. 12 a circuit structure for isolating non-signal artifacts (i.e. noise). The relationship of Signal Gain across the circuit of FIG. 12 from voltage sources 1201, 1202 to buffer amplifier 1205, can be characterized according to the function

$$SignalGain = \frac{Ri}{( Ri + Rs )} ,$$

and so Signal Gain approaches 1 as Rᵢ>>Rₛ, where Rᵢ is the resistance of resistor 1203 and Rₛ is the resistance of resistor 1204. As can be seen in FIG. 13, the relationship of noise coupling to noise frequency shows that

$${ω}_{c} = \frac{1}{2 {πC}_{c} {R}_{i}} ,$$

where ω_{c} is the noise coupling and C_{c} is a constant; a large Rᵢ results in a decreasing ω_{c} for the circuit of FIG. 12.

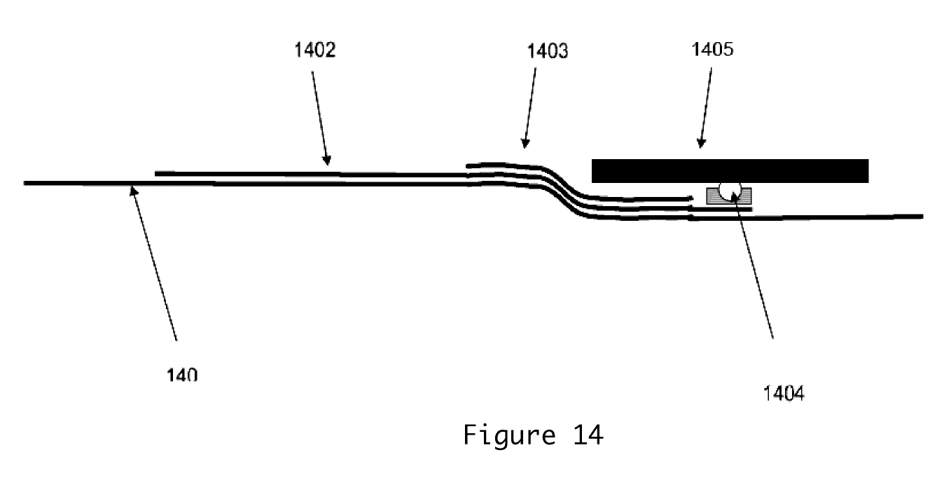

With reference now to FIG. 14, there is shown an exemplary embodiment of a sensor mounting module 1405, an articulable coupler 1404, an elastic subject-securing device 140, and a reference electrode 1402 comprising of woven conductive fabric extending from the conductive coupler 1404 within the non-conductive protective sheath portion 1403, which provides signal protection along the protected portion from non-signal artifacts and, in embodiments, provides isolation between any two electrodes.

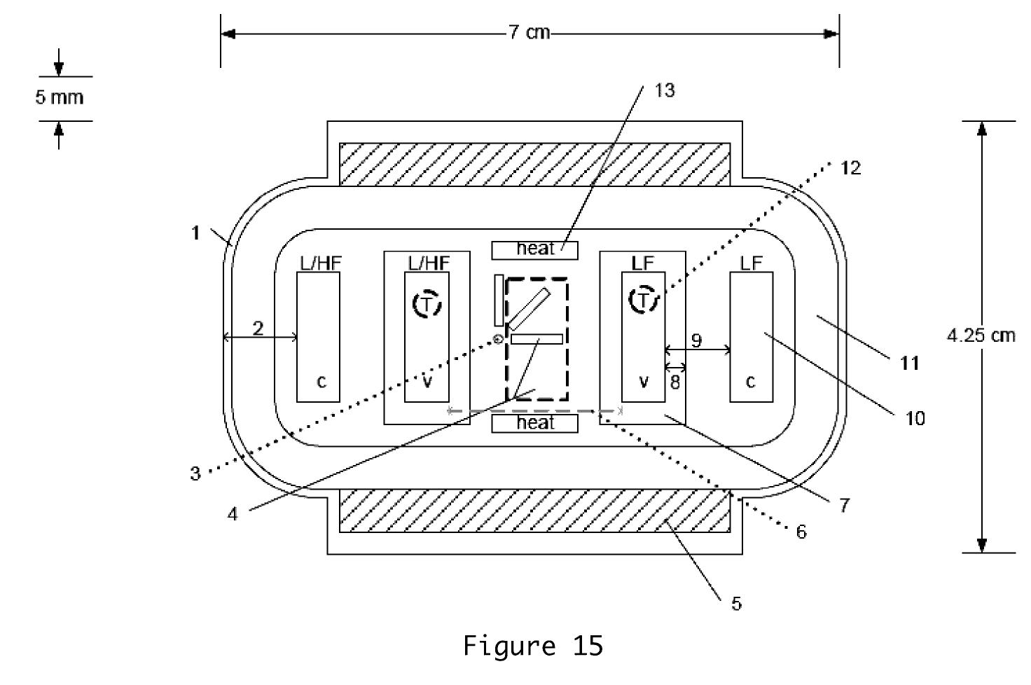

With reference to FIG. 15, there is provided an alternative exemplary sensor mounting module with a number of additional features. These include the following: On the rigid stabilizing platform 1, there is shown, the buffering region 2 (˜5 mm), an optical source 3, an optical detector 4 (e.g. a CCD OR linear array of photodiodes), moisture absorbent reservoir (indented) with absorbing material 5 for absorption of excess sweat or other fluid, a voltage electrode gap 6, a driven voltage electrode shield 7, a 1-2 mm gap 8, v-c separation 9 between current injection point (c) and voltage sensing point (v), electrodes 10 having a L/HF: Low-high frequency sensor, a LF: Low frequency, current electrode (c), and a voltage electrodes (v)), site hydration sensing module (e.g. moisturized fabric) 11, thermal sensor 12 for determining the temperature, temperature-stabilizing heaters 13 (e.g. to heat to a desired temperature using heater foil or resistance heater or to cool using a pelter cooler).

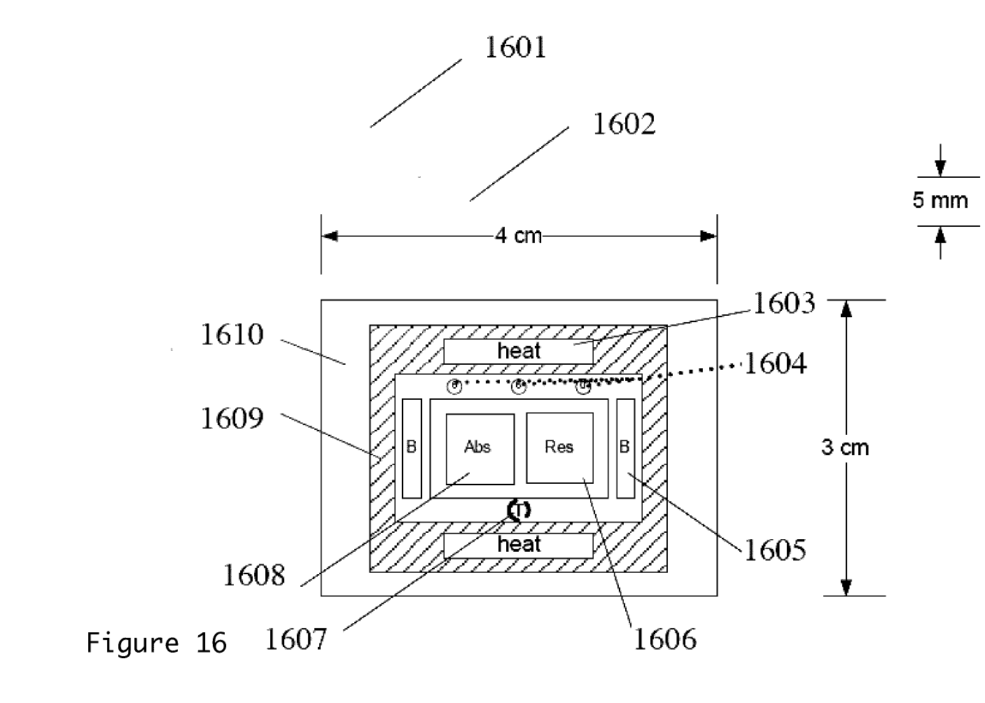

With reference to FIG. 16, there is shown an exemplary sweat sensing module. There is shown: a buffering region 1610, comprising a plastic boundary; a moisture absorbing material 1609 to absorb excess sweat and also to draw sweat across sensor through capillary action, an additional absorption pad 1608 to ensure even drawing of sweat across sensor surface, a thermal sensor 1607 for determining the temperature, a sweat collection reservoir 1606 for collecting excess sweat, bio-impedance stimulation 1605 (i.e. a signal source, comprising an A/C source), light sources 1604 (light stimulation sources e.g. via 660 nm laser or LED), sweat accentuating heater 1603 (to e.g. 40° C.), sweat-isolating layer sandwiched between device & ion-permeable layer 1602 (comprising Vaseline or oil), and an ion-permeable layer 1601 (for example, impermeability of ions that are ˜500 Daltons).

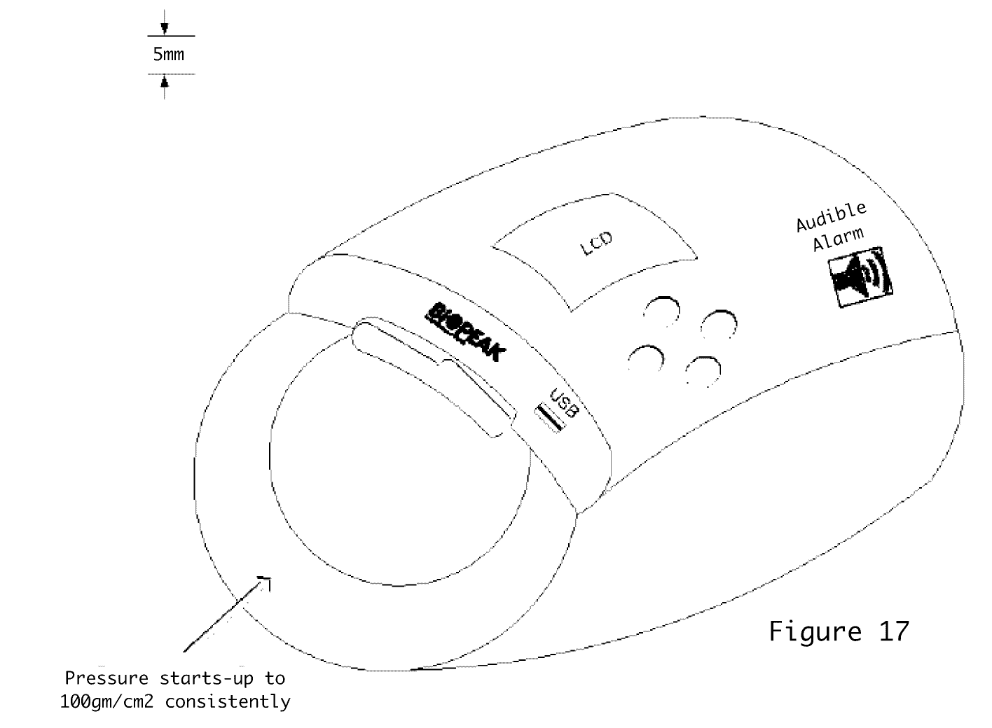

With reference now to FIG. 17, there is provided an alternative embodiment of one of the systems disclosed herein. The subject securing device is intended to secure the system to a wrist, arm or leg, rather than the trunk of a subject. The control module, in addition to the functionalities and capabilities described elsewhere herein, also comprises an interface for wired (e.g. USB port) or wireless communication, including Bluetooth or WiFi or other wireless communication apparatus and/or protocol for communicating measurements and device status, and receiving system updates and subject information, in real-time or from information stored in the memory of the control module or other portion. In addition, it includes a user input device, for inputting user information, such as medication times, exercise times, etc. It includes a rechargeable battery module, and a number of displays and audible/vibrating alarm mechanisms. Moreover, the control module comprises a memory resource for storing information related to past and current operations, as well as user-input information. In addition, it comprises a processing unit for carrying out instructions loaded as software into the memory and/or provided remotely by the communication functionalities. Such instructions may facilitate or relate to the receiving and transmission of information, the determining of calculated values based on the measured physiological signals, and the control of the various circuits, sensors and/or electrodes, and electrical components which comprise the systems and devices described herein. In some embodiments, GPS, accelerometers or other location- and motion-related measurement facilities are also located in and controlled by the control module.

An ECG subsystem in embodiments is used to pick up passive cardiac voltage potentials between an electrode on a first sensing electrode and a second sensing electrode. The raw cardiac signal is processed to determine the occurrence of R-peak. Most of the QRS complex spectrum is in the 5-30 Hz range and the ECG signal is very small, typically 4 my or less. The primary function of the circuit is to isolate the QRS complex, filter out noise, especially 50/60 Hz noise and amplify the ECG signal to a range that can be properly captured by an analog-to-digital converter (ADC) in the data acquisition subsystem. The signal is typically sampled at a rate of approximately 100 samples per second or higher. The data acquisition sub-system extracts the following data from the ECG subsystem:
R-peak using a peak detection algorithm, as described for example in G. M. Friesen, T. C. Jannett, M. A. Jadallah, S. L. Yates, S. R. Quint, and H. R. Nagle, “A comparison of the noise sensitivity of nine QRS detection algorithms”, IEEE Trans. Biomed. Eng., vol. 37, pp. 85-98, January 1990 (incorporated herein by reference);
Statistic on timing and interval of R-peaks are analyzed so that false R-peak detects and missed R-peaks are adjusted for.
Heart rate calculated from the time between R-peaks. The heart rate is typically averaged over a 5 second moving window to act as a damper to heart variability and to filter out possible invalid and missed R-peak detections.

The ECG data acquisition process may in some embodiments be designed to operate concurrently with the bio-impedance, sweat-sensor and optical/thermal/acoustic/motion data acquisition processes so that these processes can be run independently or synchronized with the ECG R-peak. The electrodes on some embodiments are permanently connected to the ECG subsystem therefore it is not necessary for the cross point switch to connect the electrodes to the ECG. Configurations without permanent ECG connections may require the electrodes to be connected to the ECG subsystem. A single ECG sample is acquired and groomed using a digital filter to be used in the R-peak search algorithm. See Friesen et al. (“A comparison of the noise sensitivity of nine QRS detection algorithms”) for a description of nine different peak search algorithms. If an R-peak is found then a time stamp is taken for use by the bio-impedance, sweat-sensor and optical/thermal/acoustic data acquisition processes for synchronization.

In some embodiments, bio-impedance is defined herein to cover the frequency range from 0 Hz to 10 MHz and RF is defined herein to cover the range from 10 MHz and higher. The bio-impedance sub-system may be used to inject alternating current into the body between electrodes on two separate sensor modules. Preferably the source supplies less than 1 mA (for safety) of sinusoidal current at several frequencies in the range of 1 Hz to 100 kHz and less than 10 mA in the range above 100 kHz. The bio-impedance subsystem measures the complex impedance across the body (between electrodes in separate sensor modules or across the local body part (between electrodes within a single sensor module). Different current levels and periodic waveforms can be used to perform a similar bio-impedance function. The resultant phase and magnitude information from the bio-impedance block is sampled by the data acquisition system so that it can be used by the signal processing function to calculate body composition information such as local and body water content, local and body electrolyte content and local and body fat content etc.

In some embodiments, the bio-impedance circuit can be connected to electrodes simultaneously with the ECG sub-system. This allows the signal processing function to use the ECG R-Peak to synchronize the bio-impedance measurements to improve the bio-impedance signal processing by focusing the processing to a specific interval in the cardiac period. The bio-impedance analysis sub-system measures the complex impedance across the body or across a local tissue area. One method of determining complex impedance is using the theory of AC phasors. By injecting a sinusoidal waveform into the body the magnitude of the complex impedance can be determined and the phase angle can be determined using a phase detector.
The current being injected into body (I_{Body}) is derived by measuring the voltage (V_{Tx}) across a series source resistor (Rₛ).

I_{Body}=V_{Tx}/R_{S}
The complex impedance magnitude of the body (Z_{Body}) is calculated by measuring the current flowing through the body (I_{Body}) and measuring the voltage drop across the body (V_{RX}) (i.e. ohm's law).

|Z_{Body}|=V_{Rx}/I_{body}
The voltage drop across the body (V_{RX}) is measured through a second set of electrodes (the reference electrodes). The electrode resistances (RE) do not affect the voltage measurement since the high input impedance of the magnitude and phase detectors draws virtually no current. In embodiments, further calibration to compensate for measurements errors such as system capacitance, gain, offset and phase errors, constant current source errors, etc can also be provided to improve overall measurement accuracy.

The phase shift (Φ_{RX}) of the injected signal with respect to the received signal can be measured using a phase detector. The real and imaginary parts of the complex impedance can be determined using the following formula:

Z Body=|Z Body|<φ_{RX}=R+jX=|Z_{Body}|cos(φ_{RX})+j|Z_{Body}|sin(φ_{RX})

The body impedance is derived from the current and voltage drop across the body. A constant current source could be used for the measurement eliminating the need to measure the current. However, in this embodiment, a measured current method is used. This method requires an additional ADC to measure the voltage drop across a reference resistor to derive the injected current. Phase can be extracted using a phase detector and is acquired through an ADC.

In some embodiments, the device may acquire at least all or part of the following data during a fixed acquisition period:
Average Impedance (Real): the average real impedance is calculated. However it may be sufficient to measure the average magnitude, which avoids having to calculate the real impedance from the raw impedance measurement.
Average Phase
Average Max (dZ/dt): This value can be synchronized with the ECG R-peak to increase the reliability of detecting dZ/dt peaks vs. other artifacts. The maximum dZ/dt typically occurs 200-400 ms through an R-peak to R-peak cycle. This dZ/dt value is averaged over the acquisition period.
Average Time from R-peak to Max (dZ/dt) if R-peak synchronization is used.

The above examples illustrate certain types of processing which can be done for a single BIA frequencies time series of acquired data. There are additional ways the data can be processed, e.g. in time domain, frequency domain, at separate BIA frequencies, interrelationships and/or combinations between multiple signals or derived signals (like ECG used to synchronize BIA processing periods as discussed in this example).

Bio-impedance can also be measured locally between electrodes in a single sensor module. The complex impedance information is used to derive local water, electrolyte and fat information. The voltage drop across the local tissue (V_{RX}) is measured through a second set of electrodes (e.g. the conductive electrodes). The electrode resistances (RE) do not affect the voltage measurement since the high input impedance of the magnitude and phase detectors draws virtually no current.

An alternative process for acquiring the bio-impedance data for local (single module) and body (multi module) measurements at a number of frequencies. First the bio-impedance electrode pairs are selected and an AC current is injected into the tissue. The injected signal is recovered and the tissue complex impedance is derived from the raw voltage, current and phase shift measurements (using ohm's law). Instantaneous and average complex impedance is recorded. Then the rate of change of the complex impedance (dZ/dt) is computed to find the maximum rate of change (max (dZ/dt)) and the time interval from R-peak to max (dZ/dt) (if R-peak synchronization is used). These values are recorded for use in the final data processing algorithms. If R-peak synchronization is used then the dZ/dt, max (dZ/dt) and timing measurements calculations are skipped unless the sample is taken during the desired time interval from R-peak. The acquisition process is repeated for each frequency and set of electrodes. The bio-impedance subsystem must wait for the injected signals to stabilize before making measurements, which makes it difficult to switch rapidly to and from the bio-impedance subsystem. For this reason the bio-impedance data acquisition process may be given an appropriate time slice to complete all of its measurements.

In some embodiments, a buffer amplifier is an electrical component that provides electrical impedance transformation from one circuit to another. Two main types of buffer exist: the voltage buffer and the current buffer. Typically a current buffer amplifier is used to transfer a current from a first circuit, having a low output impedance level, to a second circuit with a high input impedance level. The interposed buffer amplifier prevents the second circuit from loading the first circuit unacceptably and interfering with its desired operation. In the ideal current buffer in the diagram, the input impedance is zero and the output impedance is infinite (impedance of an ideal current source is infinite). Again, other properties of the ideal buffer are: perfect linearity, regardless of signal amplitudes; and instant output response, regardless of the speed of the input signal. For buffer amplifiers of the instantly disclosed subject matter, if the current is transferred unchanged (the current gain βi is 1), the amplifier is again a unity gain buffer; this time known as a current follower because the output current follows or tracks the input current. In some embodiments, a buffer may comprise sub-buffers; each sub-buffer may comprise one or more sub-buffers, in series, in parallel or both.

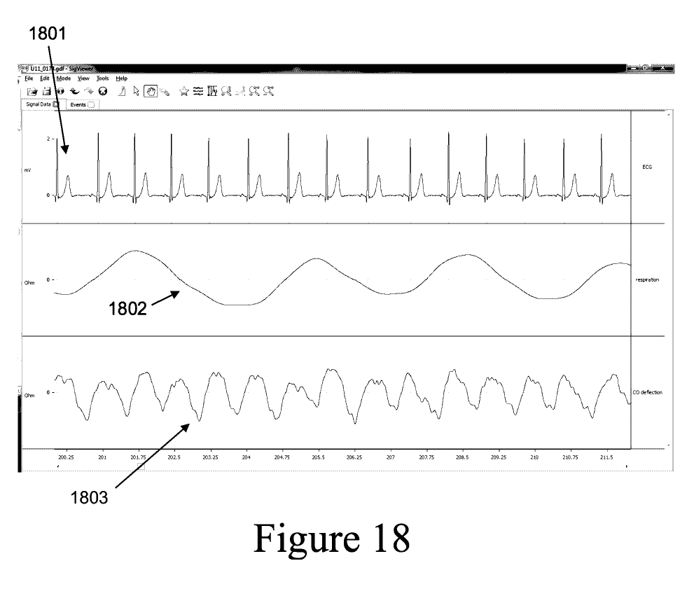

With reference to FIG. 18, there is shown an exemplary output from data collected by one aspect of the disclosed subject matter. In one embodiment, the [device] outputs information relating to ECG, respiration, and CO deflection either wirelessly (via WiFi, Bluetooth, or other wireless communication standard known in the art) or via a wired connection, and either in real-time as data is measured or afterwards as stored information after measurement. Software on a general purpose computer can output the information as an ECG output 1801, as data representative of respiration rates 1802, data representative of CO deflection 1803, or a data representative of other physiological parameters that can be measured, detected or calculated in accordance with the instantly disclosed subject matter. The data representation of the ECG values over time 1801 are determined by measuring the ECG signals and processing such signals versus time. For the data representations relating to respiration 1802 and CO deflection 1803, any one or combination of bioimpedance, acoustical, or other signals may be assessed and calculated over time to generate the data representations. In some embodiments, the device comprises a display mechanism that is configured to display one or more of the three data representations as shown in FIG. 18, or any one or more of the same or other data representations of physiological signals collected in accordance with the subject matter disclosed herein.

While the present disclosure describes various exemplary embodiments, the disclosure is not so limited. To the contrary, the disclosure is intended to cover various modifications and equivalent arrangements included within the general scope of the present disclosure.

We claim:

1. A physiological sensor system to be worn on a subject's skin to measure one or more physiological parameters, the system comprising:
at least one pair of sensing electrodes that measure, when in contact with the subject's skin, physiological signals indicative of the one or more physiological parameters;
first and second reference electrodes that concurrently provide a source signal and a reference signal for said sensing electrodes in their measurement of the one or more physiological parameters; and
a subject-securing device for securing said sensing electrodes and said first and second reference electrodes against the subject's skin in operation.

2. The system of claim 1, wherein the said physiological parameter comprises one or more of the following: a bio-impedance through the subject's body, a bio-impedance between the subject's skin and the sensing electrodes, and ECG.

3. The system of claim 1, further comprising, for at least one of the sensing electrodes, a conductive electrode, located near a perimeter of the sensing electrode and being secured against the outer surface of the subject by the subject-securing device, each conductive electrode configured to measure the impedance between each of the at least one conductive electrode and the respective sensing electrode.

4. The system of claim 1, further comprising, for at least one of the sensing electrodes, a conductive electrode, located near a perimeter of the sensing electrode, each conductive electrode configured to be driven with a shielding signal, wherein said shielding signal is in phase with the sensed signal of the respective sensing electrode.

5. The system of claim 1, further comprising, for at least one of the sensing electrodes, a conductive electrode, located near a perimeter of the sensing electrode and being secured against the outer surface of the subject by the subject-securing device, each conductive electrode connected to a current sink for parasitic current generated near the respective sensing and conductive electrode.

6. The system of claim 1, wherein a signal output of one of the sensing electrodes in at least one pair of sensing electrodes is split into a first buffer and a second buffer, the first buffer optimized for ECG signal buffering and the second buffer optimized for signal buffering of at least one of ECG and bio-impedance, the output of each being coupled to a switching circuit for selectively coupling an output signal of at least one of the first buffer and second buffer to pass through to a dual processing circuit which can be used to calculate one of ECG and bio-impedance depending on which buffer the switch is connected to.

7. The system of claim 6, wherein the switching circuit couples to the dual processing circuit the buffer that consumes less energy during times when processing the signal associated with the other buffer is not required.

8. The system of claim 1, wherein each of the at least one pair of sensing electrodes comprises an output that is an input to a buffer optimized for both ECG and bio-impedance signal buffering, the output of the buffer being split into first and second processing circuits, the first processing circuit optimized for ECG signal processing, the second processing circuit optimized for bio-impedance signal processing.

9. The system of claim 1, wherein both electrodes of at least one pair of sensing electrodes are each coupled to a buffer in close proximity to the respective sensing electrode, the output of each buffer being split, a first split from each buffer being combined into a differential input of an ECG signal processing circuit, and a second split from each buffer being combined into a differential input of a bio-impedance signal processing circuit.

10. The sensor system of claim 1, wherein the first and second reference electrodes are electrically connected to a signal ground, and the first and second reference electrodes comprise a resistor between the reference electrode and a first signal ground and the second reference electrode is electrically connected directly to a second signal ground.

11. The system of claim 1, wherein a first sensing electrode of the at least one sensing electrodes has in backside contact therewith a thermal sensor.

12. The system of claim 11, wherein the first sensing electrode further comprises at least one thermally conductive plug traversing the first sensing electrode.

13. The system of claim 1, wherein the sensor system further comprises a sweat sensor, the sweat sensor having contact with the subject's skin and being secured against the outer surface of the subject by the subject-securing device, wherein a bio-impedance is measured between the sweat sensor and another electrode.

14. The system of claim 1, wherein the sensor system further comprises an acoustical sensor, the acoustical sensor being secured against the outer surface of the subject by the subject-securing device.

15. The system of claim 1, wherein the sensor system further comprises a light source and an optical sensor, both the light source and the optical sensor being secured against the outer surface of the subject by the subject-securing device.

16. The system of claim 1, further comprising at least one minimally deformable mounting apparatus, the mounting apparatus having affixed on a subject-facing side thereof at least one sensing electrode and being urged against an outer surface of the subject by the subject-securing device.

17. The system of claim 16, wherein a perimeter of the subject facing side of at least one mounting apparatus extends beyond an outer perimeter of the sensing electrode.

18. The system of claim 16, wherein the mounting apparatus is pivotally secured on a non-subject facing side of the mounting module to the subject securing device.

19. The system of claim 1, wherein at least one of the sensing electrodes is circular with a diameter configured to provide subject contacting signal collection area with reduced susceptibility to electrode-edge induced noise artifacts.

20. The system of claim 1, wherein a buffering region structure is located near at least one edge of at least one sensing electrode.
